(19) United States
(12) Patent Application Publication
(10) Pub. No.: US 2025/0288379 A1
(43) Pub. Date: Sep. 18, 2025

(54) TRANSMISSION ASSEMBLY FOR DRIVING INSTRUMENT INSERTION, AND RELATED DEVICES, SYSTEMS, AND METHODS

(71) Applicant: INTUITIVE SURGICAL OPERATIONS, INC., Sunnyvale, CA (US)

(72) Inventor: William A. Burbank, Sandy Hook, CT (US)

(73) Assignee: INTUITIVE SURGICAL OPERATIONS, INC., Sunnyvale, CA (US)

(21) Appl. No.: 19/078,598

(22) Filed: Mar. 13, 2025

Related U.S. Application Data
(60) Provisional application No. 63/565,165, filed on Mar. 14, 2024.

(51) Int. Cl. A61B 34/00; A61B 17/29; A61B 18/14

(52) U.S. Cl. CPC A61B 34/71; A61B 18/1445; A61B 2017/2903; A61B 2017/2932; A61B 2034/715

(57) ABSTRACT

A medical instrument comprises an instrument shaft, a first movable component coupled to the instrument shaft, a first actuation element coupled to the instrument shaft, second and third actuation elements, and a transmission assembly movably coupled to the instrument shaft. The transmission assembly comprises a first drive member coupled to the actuation elements and rotatable to drive translation of the instrument shaft relative to the transmission assembly, and second drive member comprising a rotatable drive shaft and first and second actuation transfer mechanisms coupled to the rotatable drive shaft. The second actuation element is coupled to the first actuation transfer mechanism and the third actuation element is coupled to the second actuation transfer mechanisms such that the second drive member actuates the second and third actuation elements in opposite directions as one another to drive a first degree of freedom of motion of the first movable component.

### CROSS-REFERENCE TO RELATED APPLICATIONS

This application claims priority to U.S. Provisional Application No. 63/565,165, filed Mar. 14, 2024, which is incorporated herein by reference in its entirety.

### FIELD

Aspects of this disclosure relate generally to instruments for use with computer-assisted teleoperated manipulator systems, and related devices, systems, and methods. More specifically, aspects of the disclosure relate to instrument transmission assembly architectures that provide a drive for instrument insertion.

### INTRODUCTION

Some remotely-controlled instruments comprise a shaft, an end effector coupled to one end portion of the shaft, and a transmission assembly coupled along a portion of the shaft to provide forces transmitted along the shaft that control the end effector. In this way, the transmission assembly can remain outside a patient's body or other worksite region to control operations of an end effector performing functions at the desired worksite region. The end effector generally comprises one or more functional elements, such as, for example, a jaw mechanism, a stapler, a cutting implement, a camera, an electrode, a sensor, etc., to perform one or more functions of the instrument, such as cutting, sealing, grasping, imaging, etc. The transmission assembly may comprise one or more drive members configured to receive driving forces, torques, or other inputs and transmit the received driving forces, torques, or other inputs to other portions of the instrument, such as the end effector and/or other structures along portions of the instrument shaft (e.g., joints and or wrist mechanisms), to drive degrees of freedom of motion and/or functions of the instrument. The drive members may transmit received driving forces or torques to other portions of the instrument via actuation elements running from the transmission assembly along the instrument shaft. Such actuation elements may be in the form of pulling (tension) members such as cables, wires, filaments or the like that are flexible in all directions and generally transmit stronger force by pulling on the actuation element to place it in tension (sometimes referred to as pull-pull actuation elements); more rigid members such as tubes, rods, sheet metal strips, or the like that can transmit force by pushing or by pulling on the actuation element (sometimes referred to as push-pull actuation elements); semi-flexible pushing members such as push coils that can transmit force by pushing while providing lateral flexibility; rotatable members such as lead screws that can transmit rotational force; and a variety of other forms of actuation elements. In some cases, the drive members may convert received driving forces or torques from one form to another, such as, for example, converting received torques into a linear forces. Further, in some cases, combinations of the various types of actuation elements can be used depending on the nature of the force needed and/or the flexibility desired as an actuation element is routed from the transmission assembly, along the shaft, to a moveable component (such as an articulable structure and/or a component of the end effector).

The transmission assembly of such instruments can be configured to be coupled to computer-assisted teleoperated manipulator systems to receive the drive inputs, such as via servo-motor and drive disk interfaces, and/or may be configured to receive input manually, such as via a user operating a wheel, button, trigger, or other mechanism, at the transmission assembly to provide the desired input.

Some remotely-controlled instruments have a degree of freedom of motion comprising linear translation of the instrument shaft relative to a workspace (e.g., a patient's body) along a longitudinal axis of the shaft. Such translation of the instrument shaft, and hence the end effector coupled thereto, may allow for insertion of the end effector into (or advancement further into) the workspace and withdrawal of the end effector from the workspace. This degree of freedom of motion may be referred to herein as an insertion degree of freedom, and the direction along which the translation occurs may be referred to herein as an insertion axis.

Some instruments, and manipulator systems, provide for the insertion degree of freedom of motion by translating the entire instrument, including the transmission assembly, along the insertion axis. For example, for a teleoperated manipulator system, an instrument holder of the arm of a manipulator to which the instrument is removably coupled, may be translatable along a link of a manipulator of the manipulator system. Likewise, for a manually-operated instrument, insertion may occur via movement of the entire instrument including the transmission assembly, via a user generally holding the instrument at least in part by the transmission assembly housing.

However, other instruments and manipulator systems provide for the insertion degree of freedom of the instrument shaft and end effector via drive input from the transmission assembly itself, thereby causing relative translation between the shaft of the instrument and the transmission assembly. In such systems, the relative translation between the instrument shaft and the transmission assembly causes translation of the instrument shaft relative to the workspace because the transmission assembly is held translationally stationary relative to the workspace by the manipulator to which the instrument is coupled.

The ability of the instrument shaft and transmission assembly to translate relative to each other adds complexity in the architecture of drive and force transmission structures that make up the transmission assembly. There exists a need to provide instruments with transmission assemblies in which the insertion degree of freedom movement of the instrument is driven by the transmission assembly while still being relatively simple and compact in its overall architecture to provide the drive forces associated with the other functionality of the instrument, and/or to otherwise improve performance of instruments with such transmission assembly architectures.

### SUMMARY

Various embodiments of the present disclosure may solve one or more of the above-mentioned problems and/or may demonstrate one or more of the above-mentioned desirable features. Other features and/or advantages may become apparent from the description that follows.

In accordance with at least one embodiment of the present disclosure, a medical instrument may comprise an instrument shaft comprising a proximal portion and a distal portion, a first movable component coupled to the distal portion of the instrument shaft, a first actuation element coupled to the instrument shaft, a second actuation element, a third actuation element, and a transmission assembly movably coupled to the instrument shaft. The transmission assembly may comprise a first drive member, and at least one of the first, second, or third actuation elements is operably coupled to the first drive member and configured to drive translation of the instrument shaft relative to the transmission assembly in response to rotation of the first drive member. The transmission assembly may further comprise a second drive member comprising a rotatable drive shaft, a first actuation transfer mechanism coupled to the rotatable drive shaft, the second actuation element being coupled to the first actuation transfer mechanism, and a second actuation transfer mechanism coupled to the rotatable drive shaft, the third actuation element being coupled to the second actuation transfer mechanism. The first and second actuation transfer mechanisms may be configured to actuate the second and third actuation elements in opposite directions as one another to drive a first degree of freedom of motion of the first movable component.

In accordance with at least one embodiment of the present disclosure, a medical instrument may comprise an instrument shaft, a movable component coupled to a distal portion of the instrument shaft, a first actuation element coupled to the instrument shaft, a second actuation element, a third actuation element, and a transmission assembly movably coupled to the instrument shaft. The transmission assembly may comprise a first drive member comprising a drum, and each of the first, second, and third actuation elements may be at least partially wound around the drum and configured to drive translation of the instrument shaft relative to the transmission assembly in response to rotation of the drum. The transmission assembly may further comprise a second drive member comprising a drive shaft, first actuation transfer mechanism coupled to the drive shaft and to the second actuation element, and a second actuation transfer mechanism coupled to the drive shaft and to the third actuation element. The drive shaft of the second drive member may be rotatable to actuate the second and third actuation elements to drive a first degree of freedom of motion of the movable component.

In accordance with at least one embodiment of the present disclosure, a medical instrument may comprise an instrument shaft comprising a proximal portion and a distal portion, a movable component supported by the distal portion of the instrument shaft, a first pushable actuation element operably coupled to the movable component and translatable relative to the instrument shaft to drive a first degree of freedom of motion of the movable component, a plurality of pullable actuation elements, and a transmission assembly movably coupled to the instrument shaft. The transmission assembly may comprise a first drive member comprising a drum coupled to at least a subset of the plurality of pullable actuation elements and rotatable to actuate the subset of the plurality of pullable actuation elements to drive translation of the instrument shaft relative to the transmission assembly. The transmission assembly may further comprise a second drive member comprising a drive shaft rotatable to actuate a first pair of pullable actuation elements of the plurality of pullable actuation elements to drive translation of the pushable actuation element relative to the shaft.

In accordance with at least one embodiment of the present disclosure, a method of operating a medical instrument may comprise actuating a first drive member to simultaneously actuate a plurality of actuation elements coupled to the first drive member to drive relative translation between an instrument shaft and a transmission assembly of the medical instrument. The method may further comprise actuating a second drive member to simultaneously actuate a first pair of actuation elements out of the plurality of actuation elements in opposite directions to drive motion of a movable component supported by the instrument shaft, wherein the first pair of actuation elements are coupled to both the first and second drive members.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1A is a schematic diagram of an embodiment of an instrument, with various internal components illustrated.

FIG. 1B is a schematic diagram of portions of the instrument of FIG. 1A.

FIG. 1C is a schematic diagram of portions of the instrument of FIG. 1A.

FIG. 2 is a perspective view of another embodiment of an instrument.

FIG. 3 is a partial, perspective view of the instrument of FIG. 2, showing the transmission assembly housing and portion of the shaft.

FIG. 4 is a perspective view of an embodiment of a transmission assembly of the instrument of FIG. 2, from a first side thereof, and with a cover portion removed to illustrate internal components.

FIG. 5 is a schematic diagram of portions of the instrument of FIG. 2 and an arrangement of various components and actuation elements.

FIG. 6A is a plan view of an embodiment of an actuation transfer mechanism of the instrument of FIG. 2 in a first state.

FIG. 6B is a plan view of the actuation transfer mechanism of FIG. 6A in a second state.

FIG. 6C is a side view of the actuation transfer mechanism of FIG. 6A in the first state.

FIG. 6D is a side view of the actuation transfer mechanism of FIG. 6A in the second state.

FIG. 7A is a side, perspective view of a portion of the instrument of FIG. 2 with a shaft housing removed.

FIG. 7B is an enlarged perspective view of a portion of the instrument labeled 7B in FIG. 7A.

FIG. 7C is an enlarged perspective view of a portion of the instrument labeled 7C in FIG. 7A.

FIG. 8 is a perspective view of an embodiment of an end effector coupled to a distal end portion of an instrument shaft with an articulable structure.

FIG. 9 is a partial, perspective view of a portion of the end effector of FIG. 8 with a top jaw member removed.

FIG. 10 is a schematic diagram of an embodiment of a teleoperable manipulator system.

FIG. 11 is a perspective view of an embodiment of an instrument manipulating portion of a manipulator.

### DETAILED DESCRIPTION

In the description below, reference is made to pullable actuation elements and pushable actuation elements. Pullable actuation elements refers to actuation elements that comprise flexible pulling members (such as cables, wires, filaments, belts, chains, straps, ropes, etc.) that can transfer pulling (tensioning) forces while also being relatively flexible in directions perpendicular to a longitudinal dimension of the member. Pushable actuation elements refers to actuation elements that comprise more members that can transfer pushing (compression) forces, including rigid members that are capable of transmitting both pushing and pulling forces (such as rigid tubes, rods, bars, beams, etc.) and semi-flexible members that are capable of transmitting axial pushing forces but which are flexible in one or more lateral directions (such as push coils, cut tubes, laterally supported cables (e.g., so-called “push-cables”), etc.), or any combination of these.

Use of the term “pushable” should not be misconstrued as implying that the pushable actuation elements cannot also transfer pulling forces, as some pushable actuation elements may be capable of doing so (sometimes called push-pull members). In the description below, reference is also made to “paying out” or “drawing in” pullable actuation elements. Paying out a pullable actuation element refers to increasing the length of a segment of the actuation element that extends between a take-off point on a drive member to which the actuation element is coupled and a coupling point at which the actuation element is operably coupled to a moveable component whose motions is driven by the actuation element. Conversely, drawing in the pullable actuation element refers to decreasing the length of the segment of the actuation element that extends between the take-off point of the drive member and the coupling point of the driven moveable component. By extension, “paid-out length” refers to the current length of the above-described segment of the actuation element that extends between the take-off point and the coupling point. Furthermore, in the description below, reference is made to the “path length” for a pullable actuation element. The path length refers to the length of a hypothetical path that the above-described segment would be expected to traverse as it extends between the take-off point and the coupling point, assuming that the segment has no slack. The path length does not necessarily always equal the paid-out length, as some slack may be present resulting in the paid-out length exceeding the path length. References to “actuating” a pullable actuation element refers to causing the pullable actuation element to be paid out or drawn in, while references to “actuating” a pushable actuation element refer to causing the pushable actuation element to translate relative to some reference point such as the instrument shaft. References to “actuating” a drive member refer to causing the drive member to move (e.g., rotate) so as to perform some action, such as actuating an actuation element coupled thereto. The above-described terms and definitions are explained in further detail below, in and following the description of the various embodiments.

As noted above, in instruments for which an insertion degree of freedom occurs by a drive input at a transmission assembly of the instrument causing relative translation between the transmission assembly and the instrument shaft, the architecture of the transmission assembly can raise some challenges. Some aspects of one embodiment of such a transmission assembly are described briefly below to illustrate some of the challenges that may arise in such instruments. In an embodiment of a transmission assembly, one of the drive members, also referred to hereinafter as the insertion drive member, drives translation of the instrument shaft relative to the transmission assembly to achieve the insertion degree of freedom motion of the instrument shaft and end effector. Various other drive members of such a transmission assembly, also referred to hereinafter as non-insertion drive members, can each be coupled to at least one respectively corresponding actuation element in the form of pull-pull actuation elements that are actuated (drawn in or paid out) to drive a degree of freedom of motion (or other function) of the instrument. The insertion drive member may comprise a drum to which multiple pull-pull actuation elements are coupled. The actuation elements coupled to the drum include one actuation element coupled to a proximal portion of the instrument shaft to drive relative translation of the shaft and transmission assembly in one direction, and also at least some of the actuation elements that are coupled to the non-insertion drive members (in some cases, all of the actuation elements that are coupled to the non-insertion drive members). The insertion drive member and the non-insertion drive members are configured such that rotation of the insertion drive member actuates (pays out or draws in) all of the actuation elements coupled thereto independently of rotation of the non-insertion drive members, thereby causing relative translation between the shaft and transmission assembly but without consequent actuation of the movable components (e.g., end effector, wrist, etc.) coupled to the non-insertion drive members. Individual actuation elements coupled to the non-insertion drive members can also be actuated independently of the insertion drive member to cause actuation of a movable component by rotation of a corresponding one of the non-insertion drive members. In such a transmission assembly, there can be numerous actuation element segments extending between the insertion drive member and the non-insertion drive members as well as segments extending between the various drive members and the instrument shaft. It can be challenging to arrange the drive members and the actuation elements of the transmission assembly in an orderly manner that allows the cables and drive members to function as intended without tangling or otherwise interfering with one another. This can be particularly challenging when trying to design a more compact transmission assembly or when trying to include a large number of drive members or other components in the transmission assembly to facilitate driving various components of the overall instrument, such as multiple components of the end effector and/or other articulable structures, as generally speaking the smaller the transmission assembly is, or the more drive members or other components are included therein, the more likely it is that interference occurs with the actuation elements routed through the transmission assembly.

Another challenge for instruments in which the transmission assembly drives the insertion degree of freedom pertains to how to arrange and drive pushable actuation elements comprising rigid or semi-flexible push members, such as rods, tubes, push-coils, and the like. Because the transmission assembly moves relative to the shaft, various means of coupling drive inputs directly to these actuation elements may not be usable, as such coupling may result in the actuation elements moving with the transmission assembly relative to the shaft when the transmission assembly is moved along the insertion degree of freedom of motion, causing inadvertent actuation of the movable components coupled to the actuation element. Thus, it may be difficult to use rigid or semi-flexible actuation elements with a transmission assembly that drives the insertion degree of freedom.

Another challenge pertains to slack that can arise in pullable actuation elements comprising flexible pulling members when tension is not actively applied to the actuation element, such as, for example, may occur when the instrument is not mounted to a manipulator in the case of a teleoperated instrument. If sufficient slack is developed in an actuation element, it can derail from a handling component, such as a pulley or the drive members. Slack can occur as a result of movable components controlled by the actuation elements being moved while the drive members are free to rotate, such as in the unmounted state of the instrument relative to the manipulator. By way of example, a moveable component of an instrument that is coupled to actuation elements not being actively held in tension (such as due to being mounted to a manipulator and/or otherwise having drive members clocked to a degree of rotation that tightens the actuation elements) may experience an external force, for example because the instrument has accidentally been bumped against something. Because the drive members may be free to rotate (e.g., when in an unmounted state from a manipulator system or otherwise in an unlocked state), the force applied to the moveable component may cause it to move. Such movement in turn causes one of the actuation elements coupled thereto to pay out, while generating slack in the other actuation element coupled thereto. More specifically, as the moveable component moves the path length for a first actuation element increases and the moveable component pulls against the first actuation element, and because the corresponding drive member is free to rotate the first actuation element is allowed to pay out in response to the pulling. However, the same motion of the moveable component decreases the path length for a second actuation element of the pair coupled to operate the moveable component. The paid-out length of the second actuation element does not change, and therefore as the path length decreases a difference between the paid-out length of the second actuation element and the path length for the actuation element increases, resulting in slack in the second actuation element. If enough slack develops, the second actuation element may be susceptible to derailing from a handling device.

To address some of the issues noted above and otherwise improve remotely controlled instruments, some embodiments disclosed herein comprise at least one drive member that has multiple actuation transfer mechanisms driven by the same drive input of the drive member. In such a drive member, each actuation transfer mechanism thereof is coupled to a respective pullable actuation element, and when the drive input of the drive member is actuated, this results in the simultaneous actuation of all the pullable actuation elements coupled to the actuation transfer mechanisms of the drive member. For example, in some embodiments, a pair of pullable actuation elements that drives the same degree of freedom of motion of a movable component along opposite directions may be coupled to the same drive shaft of a given drive member, with the drive member being configured to actuate the pullable actuation elements in opposite directions (i.e., actuation of the drive member in one direction causes one of the actuation elements of the pair to pay out while simultaneously causing the other actuation elements of the pair to be drawn in). Thus, in such embodiments, the actuation of a single drive member may control motion of a movable component along a degree of freedom of motion, rather than having to actuate two drive members in concert with one another to control such motion. Thus, fewer drive members are needed and the transmission assembly can be made smaller, or additional parts and functionality may be added within the same size transmission assembly (e.g., the same number of drive members may be used as in prior designs, but they may drive more degrees of freedom of motion than otherwise would be possible).

In addition, in embodiments in which a pair of actuation elements that drives the same degree of freedom of motion of a movable component are coupled to and driven by the same drive member, the issue described above related to slack developing in actuation elements may be alleviated. Because the drive member is configured such that motion thereof in one direction drives actuation of the pair of actuation elements in opposite directions as one another, if one of the actuation elements ends up being inadvertently pulled to pay out a certain amount (e.g., due to the movable component being accidentally moved in an unconstrained state), this motion will result in the drive member moving in a manner causes the other paired actuation element to be drawn in by the same amount, and vice versa, thus automatically preventing (taking up) any slack that otherwise would have developed.

In addition, in some embodiments disclosed herein, pushable actuation elements comprising rigid or semi-flexible push members are coupled to the transmission assembly by more flexible pullable actuation elements, such as cables. A given pushable actuation element may be coupled to and driven by a pair of pullable actuation elements that are configured such that they are actuated (paid out or drawn in) in opposite directions to move the given pushable actuation element relative to the transmission assembly. The pair of pullable actuation elements may be coupled to the same drive member via actuation transfer mechanisms, as described above. In addition, each pullable actuation element of the pair is coupled to the insertion drive member. The pair of pullable actuation elements are wrapped in opposite directions as one another around the insertion drive member. Thus, when the insertion drive member is moved to drive relative motion between the shaft and the transmission assembly, the pair of pullable actuation elements are actuated by the insertion drive member to move in opposite directions, thus causing the pushable actuation element to move relative to the transmission assembly. The motion of the pushable actuation element relative to the transmission assembly is opposite in direction and distance as the motion of the transmission assembly relative to the shaft, resulting in the pushable actuation element staying stationary relative to the shaft. In other words, motion of the transmission assembly relative to the shaft does not affect the position of the pushable actuation elements relative to the shaft. Thus, the above-described configuration allows for pushable actuation elements to be usable even in instruments with movable transmission assemblies, thereby expanding the functionalities of the instrument. For example, in some embodiments herein, an instrument may comprise an end effector configured as a vessel sealer, which is driven by two pushable actuation elements.

Furthermore, to address some of the issues noted above and otherwise improve remotely controlled instruments, some embodiments disclosed herein provide bearing mechanisms on a drive shaft of one or more of the non-insertion drive members. The bearing mechanism provided on one drive member is configured to redirect an actuation element that is actuatable by (coupled to) another drive member as that actuation element extends between the insertion drive member and the drive member to which it is coupled. In some embodiments, the bearing mechanism may be rotatably coupled to the drive shaft such that the bearing can move with the actuation element as the actuation element is actuated, thus reducing the amount of friction that resists the actuation of the actuation element. In some embodiments in which multiple actuation transfer mechanisms are provided on the same drive shaft of one or more of the drive members, one or more of the drive members may be provided with multiple bearing mechanism on the same drive shaft to reroute the actuation elements from the drive member that has the multiple actuation transfer mechanisms. Bearing mechanisms such as those described herein allow for the drive members to be positioned in arrangements that otherwise may not be tenable. For example, if a first non-insertion drive member is positioned between the insertion drive member and a second non-insertion drive member, then the first non-insertion drive member may block or otherwise interfere with the actuation element that is actuated by the second non-insertion drive member as that actuation element extends between the insertion drive member and the second non-insertion drive member. But by providing a bearing mechanism on the shaft of the first non-insertion drive member, the actuation element of the second non-insertion drive member can be redirected around the first non-insertion drive member by the bearing mechanism, thus allowing the actuation element to be routed between the insertion drive member and the second non-insertion drive member despite the first non-insertion drive member being positioned therebetween. Accordingly, using the bearing mechanisms described herein expands the possibilities for how the drive members can be arranged relative to one another, including allowing the drive members to be positioned closer together than may otherwise be feasible. This may allow for the transmission assembly to be made more compact or to fit additional components within a given size of the transmission assembly. Moreover, because the bearing mechanism is coupled to the drive shaft of a drive member in some embodiments, as opposed to being provided separate from the drive members for example, the need to provide an additional component in the transmission assembly to support the bearing mechanism can be avoided. In addition, because the bearing mechanism is coupled to the drive shaft of the drive member in some embodiments, the bearing mechanism may fit within a footprint that would already be occupied by the drive member, thus avoiding the need to allocate additional space within the transmission assembly for the bearing mechanism and an associated supporting component.

Turning now to the figures, various embodiments will be described in greater detail. In the figures and in the description below, reference numerals are used to identify various parts, and in some cases in which multiple instances of the same or similar part are present, characters may be appended to the end of a base reference numeral to help to distinguish one particular instance of a part from another similar instance of the same or similar part. The appended characters may be omitted when the parts are being referred to generally without need to distinguish different instances of the same part. In particular, to identify different instances of the same or similar part the appended characters may take the form _N where N is any integer. For example, two drive members 400 may be referred to with the reference numbers 400_1 and 400_2 when desired to identify and differentiate specific drive members 400, or may be referred to generically by the reference number 400 when differentiation of specific instances is not desired. Moreover, the appended characters may also take the form of ‘(i.e., the apostrophe or prime character), to even further differentiate similar parts. For example, two actuation transfer mechanisms 430 of the same drive member 400_2 may be referred to with the reference numbers 430_2 and 430_2’, with the _2 indicating that the two transfer mechanisms 430 are associated with the drive member 400_2 and the ‘differentiating these two transfer mechanisms 430 from one another. In addition, letters such as a, b, c, etc. may also be appended to a reference number to indicate different portions or aspects of a part. For example, distal and proximal portions of an instrument shaft 100 may be labeled as 100a and 100b, respectively.

### First Instrument Embodiment

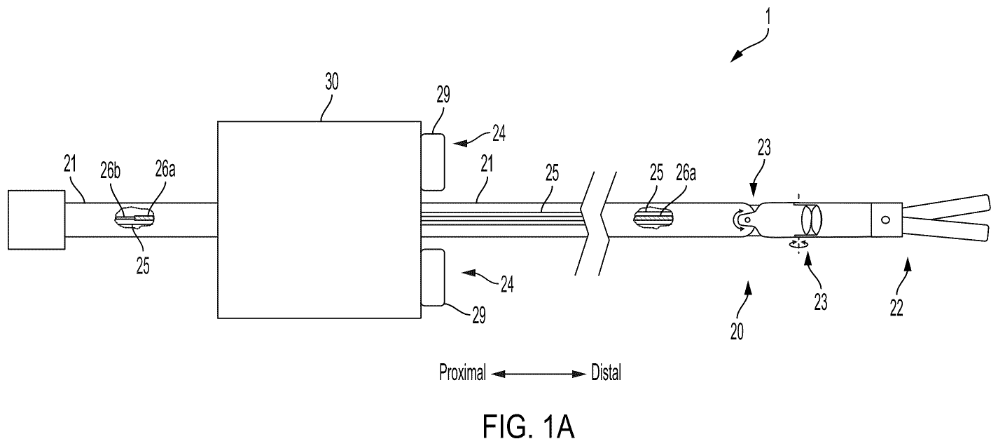

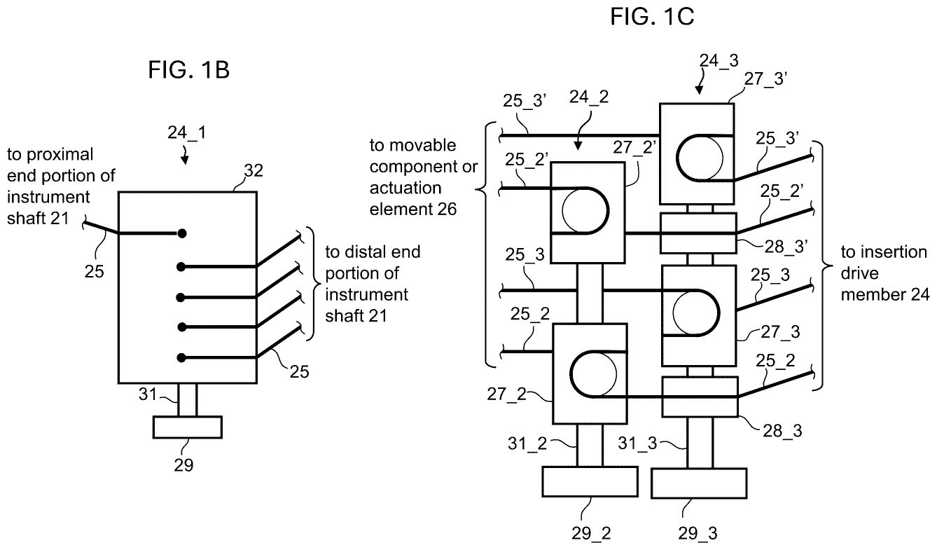

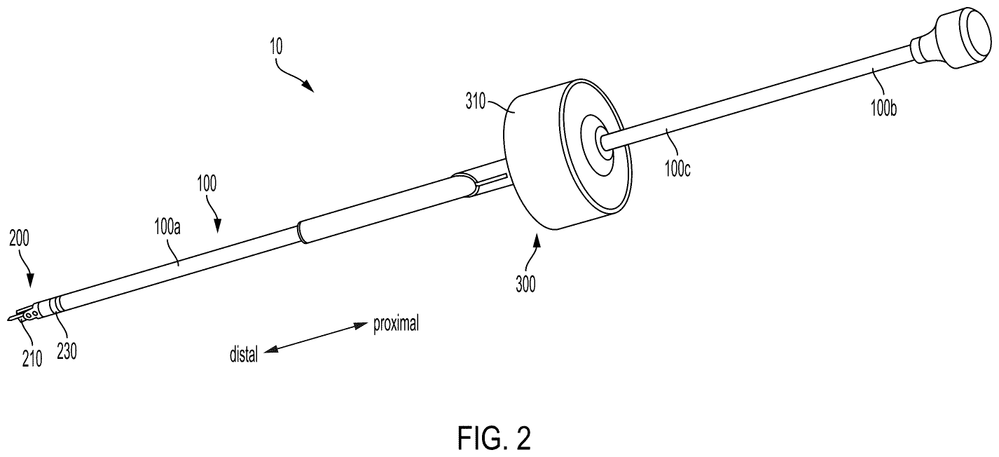

FIGS. 1A-1C illustrate an embodiment of an instrument 1 comprising a transmission assembly that drives insertion of the instrument. FIGS. 2-9 illustrates various views of another embodiment of an instrument 10. The instrument 10 may be used as the instrument 1. In some embodiments, it is contemplated that either of the instruments 1 or 10 is used with and controlled via a computer-controlled teleoperated system, such as the system 1000 described below in relation to FIG. 10. For example, the instruments 1 or 10 may be used as the instrument 1002. While various embodiments described herein contemplate an instrument configured to be mounted to a manipulator system of a computer-controlled teleoperated system, those having ordinary skill in the art would appreciate that a manually-operated instrument is also contemplated, in which case various drive members can be configured to be operated via manual drive inputs, such as wheels, buttons, and the like.

In some embodiments, the instrument 1 or 10 is a medical instrument, which may be used to perform medical procedures, such as, for example, surgical, diagnostic, or therapeutic procedures. Medical instruments may include a variety of instruments used to perform medical procedures, such as therapeutic instruments, diagnostic instruments, surgical instruments, and/or imaging instruments. In some examples, the medical instruments may be inserted into a patient through a natural orifice or an incision (including through a port or other guide inserted in the incision). Such instruments that are remotely controlled may be particularly useful, for example, in performing minimally invasive surgical procedures. A minimally invasive medical procedure may be designed to reduce the amount of tissue that is damaged during the procedure, for example by decreasing the number and/or size of incisions through which medical instruments are inserted. In other embodiments, the instrument 1 or 10 may be a non-medical instrument, such as an industrial instrument used for remote inspection or other remote procedures.

As shown in FIG. 1A, an embodiment of an instrument 1 comprises a shaft 21 supporting an end effector 22 and one or more movable components 20 at a distal end portion of the shaft 21 (proximal and distal directions referenced herein are illustrated in FIG. 1A). The end effector 22 is configured to perform one or more functions. The one or more movable components 20 may comprise one or more components of the end effector 22 that are drivable to move (e.g., a jaw member, a translating cutting component, etc.) and/or one or more articulable structures 23 (such as a wrist mechanism). The instrument 1 also comprises a transmission assembly 30, which is movably coupled to the shaft 21 proximal of the movable components 20. Because the shaft 21 and transmission assembly 30 move relative to one another, as described below, the coupling location of the transmission assembly 30 to the shaft 21 varies, but the transmission assembly 30 remains positioned generally proximal of the movable component(s) 20 of the instrument 1. In various embodiments, the transmission assembly 30 is mountable to a manipulator of a computer-controlled, teleoperated system (such as the system 1000 described in further detail below), and input driving forces are provided to the instrument through the manipulator interface (as also describe in further detail below). In other embodiments, the transmission assembly 30 is provided with inputs that allow for manual operation of the instrument 1, such as for laparoscopic medical instruments.

The end effector 22 is illustrated as having a jaw mechanism in FIG. 1A, but it should be understood that any other type of end effector could be used. Some non-limiting examples of end effectors 22 include staplers, forceps, vessel sealers, imaging devices (e.g., an endoscope tip), sensing devices (pressure, temperature, etc.) scissors, electrosurgical devices (e.g., monopolar or bipolar electrosurgical devices), other flux delivery devices (irrigation, suction, etc.), and so on. Functions performed by end effector 22 may include, for example, one or more of grasping, cutting, stapling, electrosurgical functions, illuminating, image capture, fluid delivery and/or evacuation, etc., as would be familiar to those of ordinary skill in the art. In some embodiments, one or more functions of the end effector 22 may involve motion of a movable component that is mechanically driven. In some embodiments, some functions of the end effector 22 may be driven non-mechanically, for example, by supplying electrical power or other functional flows (e.g., vacuum suction, light, etc.) to the end effector 22. Some functions of the end effector 22 may involve a combination of mechanical and non-mechanical driving inputs, such as an electrosurgical function which may comprise a mechanically driven operation of closing of a jaw member to grasp an object and an electrically driven operation of sealing or cutting the grasped object. In some embodiments, the end effector 22 does not necessarily comprise any movable components that are mechanically driven—by way of nonlimiting example, the end effector 22 may comprise an endoscope tip with an imaging device or a monopolar or bipolar electrosurgical tip member.

As shown in FIGS. 1A-1C, the transmission assembly 30 comprises drive members 24 configured to receive input driving forces via drive inputs 29, with the driving forces controlling degrees of freedom of motion of the instrument 1. The drive members 24 convert and transfer the driving forces to actuation elements 25 and 26, such as flexible pullable actuation elements 25 (e.g., cables, wires, filaments, etc.) and pushable actuation elements 26 (e.g., rods, tubes, push-coils, etc.), to drive motion of the movable components 20 or other parts of the instrument 1. The pullable actuation elements 25 may also be referred to herein as “actuation elements 25”. The pushable actuation elements 26 may also be referred to herein as “actuation elements 26”. The pullable actuation elements 25 may be coupled directly to the drive inputs 24, while the pushable actuation elements 26 may be coupled to the drive inputs 24 via some of the pullable actuation elements 25 in the manner described above. As shown in FIG. 1A, the pullable actuation elements 25 may extend distally and proximally from the transmission assembly 30 through and/or along the shaft 21, and may be coupled to the movable component 20, pushable actuation elements 26, or the instrument shaft 21. In some embodiments, the pullable actuation elements 25 may pass along an exterior of the shaft 21 for a portion thereof and then may pass through an interior of the shaft 21 along another portion thereof, as illustrated in FIG. 1A.

At least one of the drive members 24_1 (also referred to as insertion drive member(s) 24_1) is configured to drive an insertion degree of freedom of motion of the instrument (i.e., relative translation between the instrument shaft 21 and the transmission assembly 30) by actuating two or more of the pullable actuation elements 25 coupled thereto. In some embodiments, a single insertion drive member 24_1 is provided, such, as shown in FIG. 1B. In other embodiments, multiple insertion drive members 24_1 are provided and are driven in concert to control the insertion degree of freedom of motion. Some descriptions below focus on one insertion drive member 24_1 for ease of description, but a person of ordinary skill in the art would appreciate that the principles of operation described herein in relation to one insertion drive member 24_1 are applicable to multiple insertion drive members 24_1 which are driven together in concert. In particular, one of the proximally extending actuation elements 25 is coupled with an insertion drive member 24_1 and a proximal portion of the shaft 21 such that the insertion drive member 24 can drive translation of the shaft 21 in a distal direction relative to the transmission assembly 30 by drawing in this proximally extending actuation element 25. In addition, one or more of the distally extending actuation elements 25 is coupled with an insertion drive member 24_1 (the same one as mentioned above, or a different one) and with a part the instrument 1 that is distal of the transmission assembly 30, such as a movable component 20, such that the insertion drive member 24_1 can drive translation of the shaft 21 in a proximal direction relative to the transmission assembly 30 by drawing in these distally extending actuation element 25. When the transmission assembly 30 is in a fixed position, such as mounted to a manipulator or otherwise held in a fixed position, the above-noted relative translation results in translation of the shaft 21 relative to the fixed position at which the transmission assembly 30 is coupled, which can be used for insertion or withdrawal of the end effector 22 relative to the workspace. The insertion drive member 24_1 may comprise a drum, capstan, or other similar device around which actuation elements 25 are at least partially wound, such that rotation of the insertion drive member 24 actuates the actuation elements 25.

In some embodiments, each of the actuation elements 25 is coupled to an insertion drive member 24_1. For example, in some embodiments all of the actuation elements 25 are coupled to the same single insertion drive member 24_1. Thus, in such embodiments, actuation of the single insertion drive member 24_1 causes simultaneous actuation of all of the actuation elements 25. In other embodiments one or more of the actuation elements 25 are coupled to one insertion drive member 24_1 while one or more others of the actuation elements 25 are coupled to one or more other insertion drive members 24_1. In some of these embodiments, the insertion drive members 24_1 are all driven simultaneously together to actuate the insertion degree of freedom of motion, although the insertion drive members 24_1 are not necessarily rotated in the same direction as one another (the direction of rotation of each insertion drive member 24_1 will depend on how the actuation elements 25 are wrapped around the insertion drive members 24_1 and the desired direction of actuation of the actuation elements coupled thereto).

In some embodiments, less than all of the actuation elements 25 are coupled to an insertion drive member 24_1. For example, in some embodiments a subset of the actuation elements 25 is coupled to the same single insertion drive member 24_1 and a remainder of the actuation elements 25 are not coupled to any insertion drive member 24_1. As another example, in some embodiments a first subset of the actuation elements 25 is coupled to one insertion drive member 24_1, a second subset of is coupled to one or more other insertion drive members 24_1, and a third subset is not coupled to any insertion drive member 24_1. As used herein a “subset” refers to one or more members of a collective.

In addition to the insertion drive member(s) 24_1 described above, one or more other drive members 24 (also referred to as non-insertion drive members 24) are configured to drive additional degrees of freedom of motion by actuating one or more respectively corresponding actuation elements 25 coupled thereto. FIG. 1C illustrates two examples of non-insertion drive members 24_2 and 24_3. As shown in FIG. 1C, each of the non-insertion drive member 24_2 and 24_3 comprises at least a drive shaft 31 coupled to an actuation element handling device 27. The actuation element handling device 27 is coupled to a corresponding actuation element 25 such that rotation of the drive shaft 31 actuates (draws in or pays out) the corresponding actuation element 25. The actuation element handling device 27 may comprise, for example, a capstan, drum, pulley, shaft, gear, or any other mechanism capable of actuating an actuation element 25 by rotating. An actuation element 25 may be coupled to an actuation element handling device 27 by, for example, at least partially winding the actuation element 25 around the actuation element handling device 27. In some embodiments, a drive input 29 is coupled to the drive shaft 31 to drive rotation thereof. The drive input 29 receives input driving forces, for example which may be received from a manipulator or from manual input. FIG. 1C illustrates conceptually an example configuration of actuation elements 25 coupled to example actuation handling devices 27, but embodiments of the disclosure are not limited to this configuration. In particular, the manner depicted in FIG. 1C in which the actuation elements 25 are coupled to the actuation element handing devices 27, including the directions the actuation elements 25 are wound around the actuation element handling devices 27, is illustrative only and the actuation elements 25 can be coupled to the actuation element handing devices 27 differently in other embodiments.

In some embodiments, at least one of the non-insertion drive members 24 comprises multiple (e.g., 2 or more) of the actuation element handling devices 27 such that the drive member 24 can actuate multiple actuation elements 25 simultaneously. For example, in FIG. 1C the illustrated non-insertion drive members 24_2 and 24_3 each have a pair of two actuation element handling devices 27 and 27’ coupled to the same drive shaft 31. More specifically, the drive member 24_2 has actuation element handling devices 27_2 and 27_2′ coupled to its drive shaft 31_2, while the drive member 24_3 has actuation element handling devices 27_3 and 27_3′ coupled to its drive shaft 31_3. The actuation elements 25_2 and 25_2′ are coupled to the actuation element handling devices 27_2 and 27_2′, respectively, and the actuation elements 25_3 and 25_3′ are coupled to the actuation element handling devices 27_3 and 27_3′, respectively. In some embodiments, the actuation elements 25_2 and 25_2′ drive the same movable component as one another along a same degree of freedom in opposite directions, and similarly the actuation elements 25_3 and 25_3′ actuation elements 25_2 and 25_2′ drive a same movable component as one another along a same degree of freedom in opposite directions. In some embodiments, the actuation elements 25_2 and 25_2′ are coupled to the actuation element handling devices 27_2 and 27_2′ in reversed configurations such that motion of the drive member 24_2 in one direction actuates the pair of actuation elements 25_2 and 25_2′ in opposite directions (e.g., rotation of the drive member 24_2 in one direction results in paying out one actuation element 25_2 and 25_2′ while simultaneously drawing in the other actuation element 25_2 and 25_2′, and vice versa for rotation of the drive member 24_2 in an opposite direction). The same may also be true for the actuation elements 25_3 and 25_3′ and the drive member 24_3. In some embodiments, all of the non-insertion drive members 24 comprise multiple actuation element handling devices 27, like the two non-insertion drive members 24_2 and 24_3 that are illustrated in FIG. 1C. In other embodiments, only one or some of the non-insertion drive members 24 comprise multiple actuation element handling devices 27. In some embodiments, the actuation element handling devices 27 may be configured as actuation transfer devices, which are configured to allow an actuation element 25 coupled thereto to pass through the actuation element handling device 27 to also couple with the insertion drive member 24 such that the actuation element 25 can be independently actuated by both the insertion drive member 24 and the non-insertion drive member 24 of which the actuation element handling device 27 is a part.

For ease of understanding, FIG. 1C illustrates an example of how the actuation elements 25 are coupled to and wound around the actuation element handling devices 27

The actuation elements 25 driven by the non-insertion drive members 24 may be coupled to corresponding movable components 20 to drive motion thereof. These actuation elements 25 may be coupled directly to a corresponding movable component 20, or they may be coupled indirectly to a corresponding movable component 20, for example via one of the pushable actuation elements 26. For example, in some embodiments, two pushable actuation elements 26 are provided, namely a first pushable actuation element 26a and a second pushable actuation element 26b, as shown in FIG. 1A, and each of these pushable actuation elements 26a and 26b may be coupled to and driven by a corresponding pair of the actuation elements 25. In some embodiments, a pair of actuation elements 25 that drives a given actuation element 26a or 26b may be coupled to and actuatable by the same drive member 24. The pair of actuation elements 25 that drive a given actuation element 26a or 26b may also be coupled to the insertion drive member so as to be actuated thereby in opposite directions as one another, thus allowing the actuation element 26a or 26b to remain stationary relative to the instrument shaft 21 when the transmission assembly 30 and shaft 21 translate relative to one another, as described above. In some embodiments, the actuation element 26b passes through an interior channel within the actuation element 26a, as shown in FIG. 1A. In other embodiment, the actuation elements 26a and 26b may be positioned side by side. In some embodiments, the actuation elements 26a and 26b drive motions of movable components 20 that are part of the end effector 22.

In some embodiments the one or more movable components 20 of the instrument 1 include one or more articulable structures 23 (such as jointed links, flexible portions of a shaft, etc.). Each articulable structure 23 has at least one corresponding degree of freedom of motion, which is driven by one or more actuation elements 25 coupled to the articulable structure 23. The articulable structure 23 can be used to couple the end effector 22 to the shaft 21 to allow for relative motion between the end effector 22 (or some other component) and the shaft 21, thereby allowing the pose of the end effector 22 relative to the shaft 21 to be changed (for example in degrees of freedom corresponding to pitch and/or yaw). Such placement for an articulable structure 23 is nonlimiting and articulable structures 23 can be used along the instrument shaft 21 to provide differing poses of portions of the shaft relative to other portions of the shaft 21, as those having ordinary skill in the art would be familiar with. In some embodiments, the articulable structures 23 provides differing degrees of freedom of motion. In some embodiments, multiple articulable structures 23 having differing degrees of freedom of motion are connected in series to form a wrist mechanism that couples the end effector 22 to the shaft 21 and to enable the end effector 22 to move with two or more degrees of freedom of motion (e.g., yaw, pitch, or combinations thereof) relative to the shaft 21. In some embodiments, the end effector 22 is coupled directly to the shaft 21 without an intervening articulable structure 23. References herein to an end effector being coupled to or supported by a shaft should be understood as broadly including both direct coupling and indirect coupling (e.g., via an articulable structure 230 or other intervening component), unless otherwise indicated or implied by the context. Although two articulable structures 23 comprising rotating joints are illustrated in FIG. 1A, it should be understood that this is not limiting and any number or type of articulable structure 23 could be used instead, including zero, one, three, or more articulable structures 23.

Moreover, in some embodiments, the one or more movable components 20 of the instrument 1 comprise movable components of the end effector 22 (in addition to, or in lieu of the articulable structures 23). In such embodiments, the movable components of the end effector 22 may be mechanically driven by driving forces transmitted to the end effector 22 from the drive members 24 via the pushable actuation elements 26 as already described above and/or by the flexible pullable actuation elements 25. Examples of such movable components of an end effector 22 may include a translatable blade, a pivotable jaw member of a jaw mechanism, a translatable staple firing shuttle of a stapler, etc. Such movable components of the end effector 22, when present, are instances of the above-noted movable components 20, and their motion may be considered a degree of freedom of motion of the instrument 1.

The instrument 1 may also have additional degrees of freedom of motion. For example, in some embodiments the shaft 21 may be rotatable about its longitudinal axis relative to the workspace. For example, in some embodiments such rotation of the shaft 21 relative to the workspace may be achieved by rotation of the entire instrument 1 about the longitudinal axis. This may occur, for example, by rotation of an instrument holder to which the instrument 10 is mounted relative to a support that the instrument holder is coupled with (and hence rotation of the instrument 1 as a whole relative to the support). Such rotation of the instrument shaft 21 may be referred to as a roll degree of freedom of motion.

In some embodiments, the instrument 1 may also comprise a bearing mechanism for routing an actuation element in the transmission assembly 30. As shown in FIG. 1C, a bearing mechanism 28 is provided on a drive shaft 31 of one or more of the non-insertion drive members 24. The bearing mechanism 28 provided on one drive member 24_3 is configured to redirect an actuation element 25 that is actuatable by (coupled to) another drive member 24_2 as that actuation element 25 extends between the insertion drive member 24_1 and the drive member 24_2 to which the actuation element 25 is coupled. In some embodiments, the bearing mechanism 28 may be rotatably coupled to the drive shaft such that the bearing mechanism 28 can move with the actuation element 25 as it is actuated, thus reducing the amount of friction that resists the actuation of the actuation element 25. In some embodiments, two (or more) bearing mechanism 28 may be provided on a given drive member 24 to reroute multiple actuation elements 25 from other drive members 24. For example, in FIG. 1C drive member 24_3 shown on a right side in the figure has a pair of bearing mechanisms 28_3 and 28_3′, with the bearing mechanism 28_3 being configured to reroute an actuation element 25_2 extending from the actuation element handling device 27_2 of the drive member 24_2 shown on the left side of the figure, and a second bearing mechanism 28_3′ configured to reroute an actuation element 25_2′ extending from the actuation element handling device 27_2′ of the left drive member 24_2.

Although FIGS. 1A-1C illustrate an embodiment having all of the above-described components together for ease of description, it should be understood that embodiments with different combinations of these parts are contemplated herein. In particular, embodiments contemplated herein include embodiments that have the multiple actuation element handling devices 27 on the same drive member 24 but do not have the bearing mechanisms 28 or the pushable actuation elements 25, embodiments that have multiple actuation element handling devices 27 on the same drive member 28 and the bearing mechanisms 28 but do not have the pushable actuation elements 25, embodiments that have multiple actuation element handling devices 27 on the same drive member 24 and the pushable actuation elements 25 but do not have the bearing mechanisms 28, embodiments that have the pushable actuation elements 25 but do not have the multiple actuation element handling devices 27 on the same drive member 24 or the bearing mechanisms 28, and embodiments that have the pushable actuation elements 25 and the bearing mechanisms 28 but do not have the multiple actuation element handling devices 27 on the same drive member 24. Other combinations of the various components are also contemplated herein, as would be apparent to a person of ordinary skill in the art.

### Second Instrument Embodiment

Turning now to FIGS. 2-9, another embodiment of an instrument 10 is described below. The instrument 10 may be used as the instrument 1. The descriptions above of parts of the instrument 1 are applicable mutatis mutandis to the similar parts of the instrument 10.

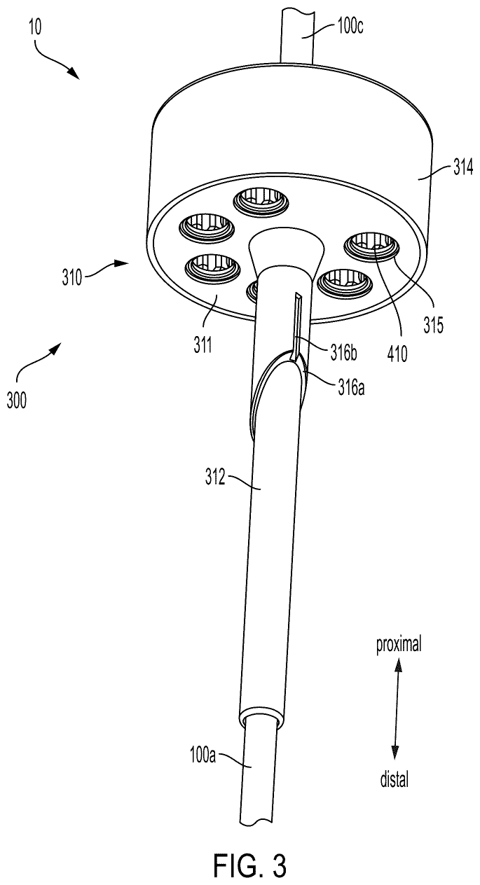

As shown in FIGS. 2 and 3, an embodiment of an instrument 10 comprises a shaft 100 supporting an end-effector 210 at a distal end portion of the shaft 110 (proximal and distal directions referenced herein are illustrated in FIGS. 2 and 3). The end effector 210 is configured to perform one or more functions, similar to the end effector 22 as described above. The instrument 10 also comprises one or more movable components 200, similar to the movable components 20 described above, coupled to a distal end portion of the shaft 100. The one or more movable components 200 may comprise one or more components of the end effector 210 (e.g., a jaw member of a jaw assembly, a translating component such as a cutting component, etc.) and/or one or more articulable structures 230 coupling the end effector 210 (or some other component of the instrument 10) to the shaft 100 and/or provided along the shaft 100 to impart articulation to the shaft 100. The instrument 10 also comprises a transmission assembly 300, similar to the transmission assembly 30 described above, which is movably coupled to the shaft 100 proximal of the movable components 200 (the shaft 100 and transmission assembly 300 move relative to one another, as described below, and thus the coupling location of the transmission assembly 300 to the shaft 100 varies, but the transmission assembly 300 remains positioned generally proximal of the movable component(s) 200 of the instrument 10).

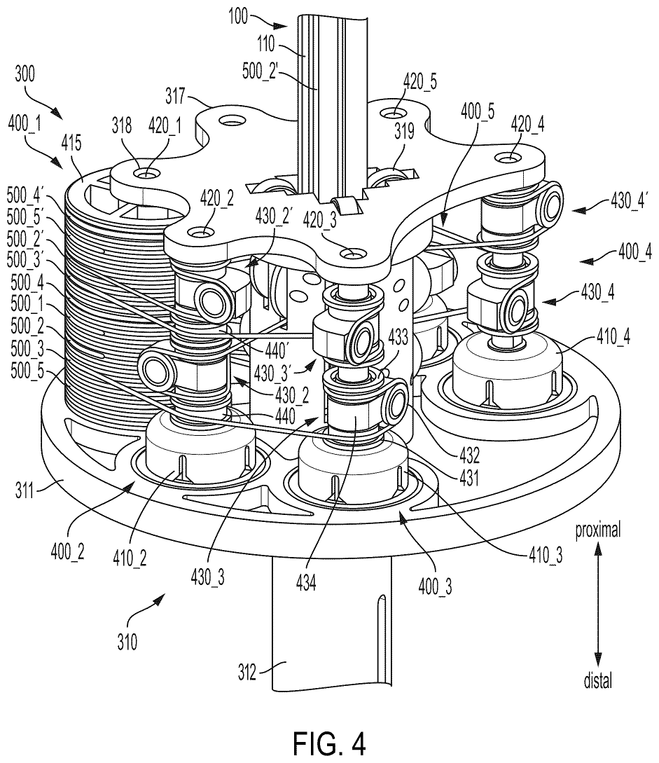

The transmission assembly 300 may be used as the transmission assembly 30, and comprises drive members 400 (described in greater detail below in relation FIG. 4) configured to receive input driving forces, with the driving forces controlling degrees of freedom of motion of the instrument 10. In various embodiments, the transmission assembly 300 is mountable to a manipulator of a computer-controlled, teleoperated system (such as the system 1000 described in further detail below), and the input driving forces are provided to the drive members 400 through the manipulator interface (as also describe in further detail below). The drive members 400 may be used as the drive members 24 described above. The drive members 400 convert and transfer the driving forces to actuation elements, including pullable actuation elements 500 and pushable actuation elements 150 and 160. FIGS. 4, 5 and 7A-7C, which are described in greater detail below, illustrate the pullable actuation elements 500 and the pushable actuation elements 150 and 160.

The pullable actuation elements 500 may be used as the pullable actuation elements 25 described above, and may also be referred to herein as “actuation elements 500.” The pullable actuation elements 500 extend through and/or along the shaft 100 and are coupled to the movable components 200 and/or other parts of the instrument 10 to transmit the driving forces so as to drive various degrees of freedom of motion of the instrument 10, which may include degrees of freedom of motion of the movable components 200 as well as additional degrees of freedom of motion.

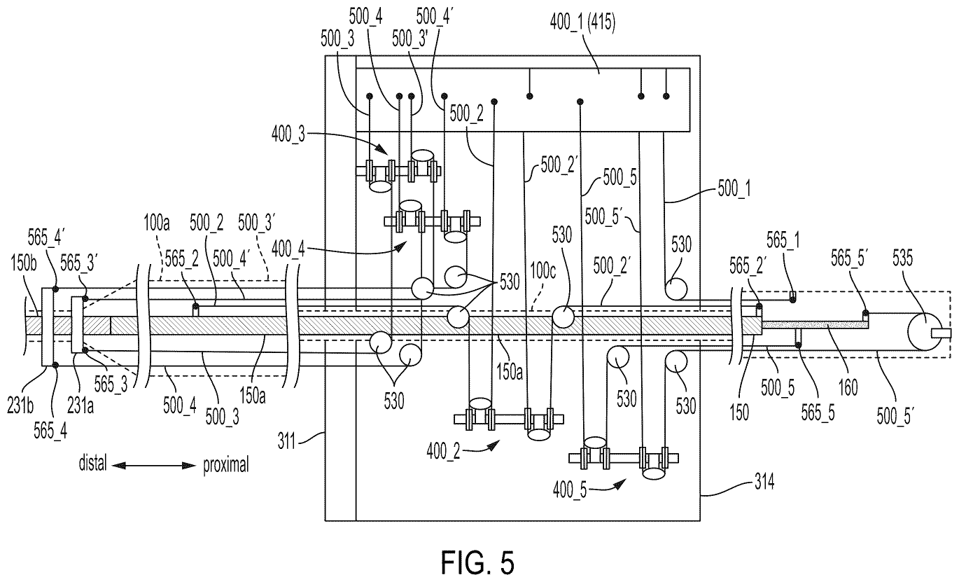

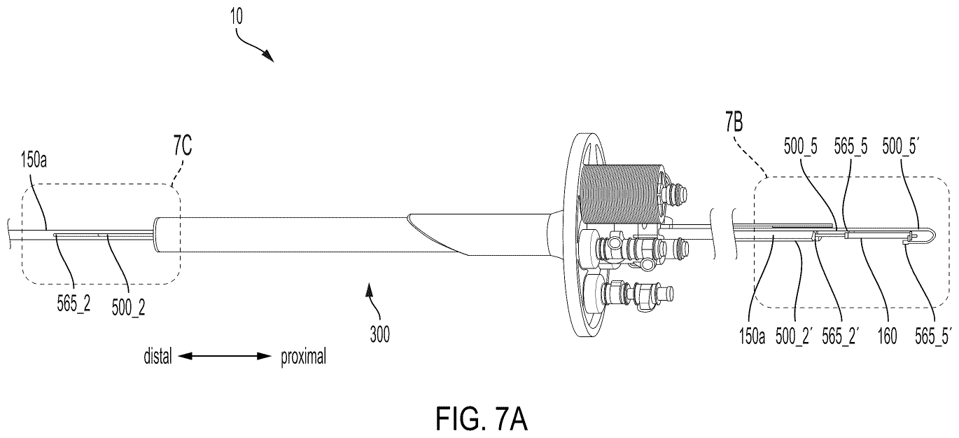

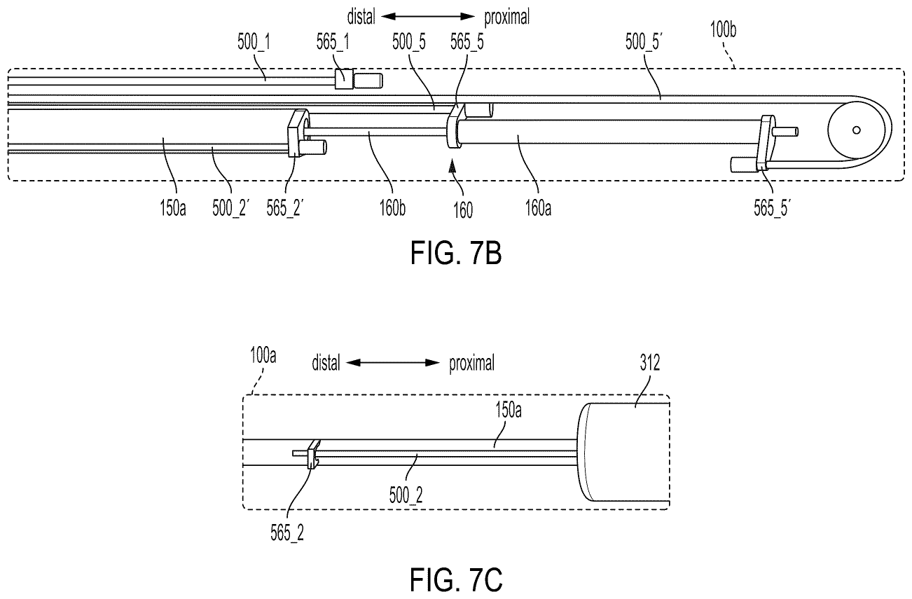

Specifically, some of the actuation elements 500 are coupled to and drive the pushable actuation elements 150 and 160 (see FIGS. 5 and 7A). Others of the actuation elements 500 are coupled to and drive motions of articulable elements 230 (such as jointed links, flexible portions of a shaft, etc.), each of which has at least one corresponding degree of freedom of motion. Still other actuation elements 500 may be coupled to the shaft 100, for example to drive an insertion degree of freedom of motion.

The pushable actuation elements 150 and 160 may also be referred to herein as “actuation element 150” or “actuation element 160”, respectively. In some embodiments, one or both of the actuation elements 150 and 160 may have a multi-part structure with one or more parts comprising a more rigid push member (such as a tube, rod, or the like) and one or more parts comprising a semi-flexible push member (such as a coil pipe, cut tube, laterally supported cable, or the like). In particular, in some embodiments at least a portion of the actuation elements 150 and/or 160 that passes through an articulable structure 230 is configured as a semi-flexible member, which allows the actuation element s150 and/or 160 to bend laterally with the articulable structure 230 (e.g., joint) while retaining the ability to transfer axial pushing forces even while bent. In some embodiments, the actuation element 150 comprises a central channel through which the actuation element 160 extends. In some embodiments, the actuation element 150 comprises a push coil or cut tube (at least while traversing articulable structures 230), and the actuation element 160 extends through a central channel of the push coil. In some of these embodiments, a portion of the actuation element 160 that extends through the central channel of the push-coil or cut tube portion of the actuation element 150 comprises a flexible pullable type member, such as a cable for example, with the actuation element 160 laterally supporting the flexible member of the actuation element 150 to reduce buckling and thereby allowing the flexible pullable member to act as a semi-flexible pushable member. In some embodiments, the actuation elements 150 and 160 are coupled to and drive motions of movable components of the end effector 210, such as a jaw member, a translating component (e.g., cutting element), etc. The pushable actuation elements 150 and 160 may be used as the actuation elements 26a and 26b described above.

As noted above, some portions of the flexible pullable actuation elements 500 may be routed inside the instrument shaft 100 and other portions may be routed external to the instrument shaft 100. More specifically, in some embodiments the actuation elements 500 may be routed inside of the instrument shaft 100 while passing through a distal portion 100a and a proximal portion 100b of the shaft 100 (see FIGS. 2 and 5), and the actuation elements 500 may be routed along an exterior of the instrument shaft 100 while traversing an intermediate portion 100c of the shaft 100, the intermediate portion 100c being located between the distal and proximal portions 100a and 100b. The intermediate portion 100c may comprise a portion of the instrument shaft 100 along which the transmission assembly 300 translates. In particular, the actuation elements 500 may transition from the exterior of the instrument shaft 100 to an interior thereof via openings (not illustrated) in and through an outer wall of the instrument shaft 100.

Components of the transmission assembly 300 are described below in greater detail with reference to FIGS. 2-9. For organizational purposes and to assist in readability, the description includes subheadings for the various components that will be described. However, it should be understood that while in some embodiments, the various components are used together, other embodiments of force transmission assemblies may not necessarily include all the components discussed.

### Transmission Assembly Housing

As shown in FIGS. 2 and 3, the transmission assembly 300 comprises a housing 310. The housing 310 comprises a chassis 311, a sleeve 312 extending distally from the chassis 311, and a cover portion 314. The chassis 311 serves as a platform or a base that supports other components of the transmission assembly 300, such as the drive members 400 described in further detail below. The chassis 311 and the cover portion 314 cooperate together to define a chamber in the housing 310 within which the drive members 400 and other components of the transmission assembly 300 are housed.

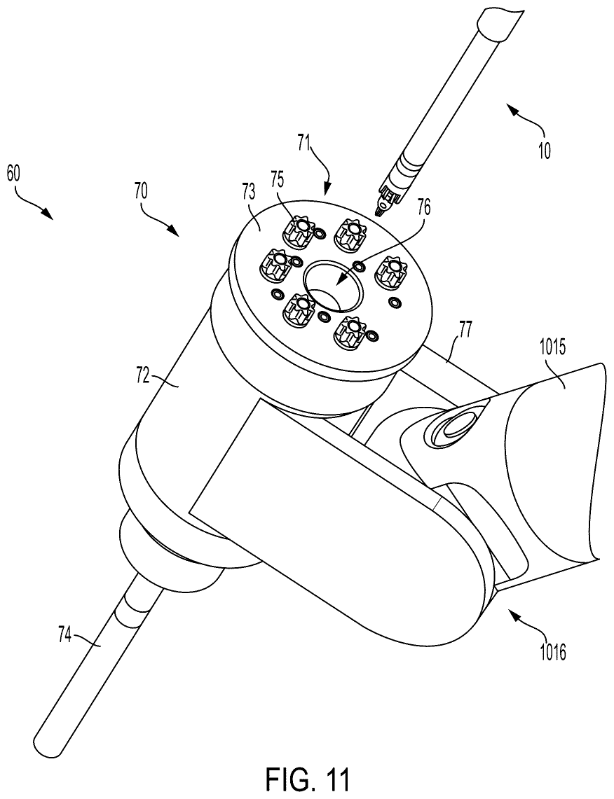

In the embodiment of FIGS. 2-9, the chassis 311 can provide an interface for coupling the instrument 10 to a manipulator in embodiment in which the instrument is configured for use with a teleoperated manipulator system. Specifically, a distal face of the chassis 311, visible in FIG. 3, forms an interface for the instrument 10 that is mountable to a complementary interface of the manipulator (e.g., interface 73, described below with respect to FIG. 11). The chassis 311 may engage either directly with the manipulator, or the chassis 311 may engage indirectly with the manipulator via an intermediary device, such as a sterile adaptor that is interposed between the manipulator and the instrument 10 to provide a sterile barrier, as those have ordinary skill in the art would be familiar with. As shown in FIG. 3, the distal face of the chassis 311 comprises openings 315 through which drive inputs 410 of the drive members (visible in FIGS. 4, 5 and 8) are accessible from an exterior of the chassis 311, for example from the distal side of the chassis 311 in the depicted embodiment. Corresponding drive outputs of the manipulator to which the instrument 10 is mounted (e.g., drive outputs 75, described below with respect to FIG. 11) may thus interact with the drive inputs 410 via the openings 315 to transfer driving forces to the drive inputs 410. The drive outputs of the manipulator may engage directly with the drive inputs 410, or they may be engaged indirectly via an intermediary, such as intermediate couplers of a sterile adaptor. The drive inputs 410 and the drive outputs (or intermediate couplers) may have complementary engaging surfaces, such as engaging surfaces with complementary groves and protrusions or other complementary keying features, such that when the drive inputs 410 are engaged with the drive outputs or intermediate couplers they are constrained to rotate together. In FIG. 3, the drive inputs 410 are shown as having receptacles into which complementary drive outputs or intermediate couplers are received, but in other embodiments, the drive inputs 410 may comprise protrusions configured to be received within complementary receptacles of the drive outputs or intermediate couplers, or any of a variety of other mating engagements to allow the drive inputs to engage with the corresponding drive outputs or intermediate couplers. In FIG. 3, the drive inputs 410 are shown as being wholly contained within the housing 310, but in other embodiments the drive inputs 410 may extend distally from the chassis 311 through the openings 315.

A sleeve 312 of the housing 310 surrounds the instrument shaft 100 and extends distally from the chassis 311. The sleeve 312 is configured to be insertable into a passage of a manipulator when the instrument 10 is mounted to the manipulator. The sleeve 312 facilities sealing of the workspace to prevent escape of pressurized fluid. The sleeve 312 may also comprise alignment features 316 to aid in alignment of the instrument 10 relative to the manipulator as the instrument 10 is mounted to the manipulator. For example, FIG. 3 illustrates two alignment features 316, namely first alignment feature 316a and second alignment feature 316b. The first alignment feature 316a comprises an angled surface portion forming a shoulder or series of shoulders extending circumferentially around the sleeve 312 and configured to engage with complementary surfaces of the manipulator as the sleeve 312 is inserted into the passage of the manipulator to provide progressively increasing alignment between the instrument 10 and the instrument holder the further the instrument 10 is inserted into the passage. The second alignment feature 316b may comprise one or more straight notches extending proximally from an apex of the first alignment feature 316a and generally parallel to a longitudinal axis of the instrument shaft 100. The second alignment feature 316b interacts with a complementary feature of the manipulator once the instrument 10 has been inserted sufficiently far into the passage. The first alignment feature 316a is configured such that they will bring the instrument 10 into an aligned orientation by the time the instrument 10 has advanced sufficiently into the passage for the second alignment features 316b to be engaged. The second alignment feature 316b then holds the instrument 10 in the aligned orientation as the instrument 10 continues to be advanced into the passage until a mounted position is reached. The initial alignment forced by the first alignment feature 316a may bring the drive inputs 410 into alignment with the corresponding drive outputs of the manipulator prior to coupling thereof, and then the continued advancement of the instrument thereafter while the second alignment feature 316b maintains the aligned orientation may enable the coupling between the drive inputs 410 and drive outputs to be completed.

In the illustrated embodiment, the sleeve 312 and the chassis 311 are integrally coupled together, or in other words they are both part of the same monolithic body. In other embodiments, the sleeve 312 and the chassis 311 are separately formed parts that are coupled together, for example, by mechanical fasteners, friction fitting, adhesive, welding, or other known joining techniques.

### Drive Members—Overview

Turning now to FIG. 4, embodiments of drive members 400 and other related components of the transmission assembly 300 will be described in greater detail. FIG. 4 comprises a perspective view of the transmission assembly 300, with the cover portion 314 removed to show the drive members 400 and actuation elements 500. The drive members 400 may be used as the drive members 24 described above.

As shown in FIG. 4, the transmission assembly 300 comprises a plurality of drive members 400. The drive members 400 each actuate (i.e., pay out or draw in) one or more actuation elements 500, which drives a corresponding degree of freedom of motion of the instrument 10. In the embodiment illustrated in FIG. 4, each drive member 400 actuates two actuation elements 500. As noted above, the drive members 400 are operably coupled to actuate pullable actuation elements 500 (e.g., a cable, wire, filament, etc.). Some of these pullable actuation elements 500 are coupled to and actuate pushable actuation elements 150 and 160, and thus the drive members 400 that drive those actuation elements 500 may also be said to actuate the pushable actuation elements 150 and 160 (via actuation of the pullable actuation elements 500 coupled to the pushable actuation elements 150 and 160). In the illustrated embodiment, the transmission assembly 300 comprises five drive members 400, with a first drive member 400_1 being configured to drive an insertion degree of freedom of motion and the other drive members 400_2 to 400_4 being configured to drive at least one other degree of freedom of motion of the instrument 10. The drive member 400_1 may also be referred to herein as an “insertion drive member,” and the other drive members 400 may also be referred to as “non-insertion drive members.” Although four non-insertion drive members 400_2 to 400_5 are illustrated, it should be understood that any number of non-insertion drive members equal to or greater than one may be included.

As noted above, each drive member 400 drives a corresponding degree of freedom of motion of the instrument 10 by paying out or drawing in at least one corresponding actuation element 500. In particular, as shown in FIG. 4, each drive member 400 comprises a drive shaft 420 about which the drive member 400 is rotatable relative to the chassis 311, with rotation of the drive member 400 about its drive shaft 420 causing the corresponding actuation elements 500 to pay out or draw in, depending on the direction of rotation. Paying out or drawing in one of the actuation elements 500 may also be referred to herein generically as “actuating” the actuation element 500 when the particular direction of motion (i.e., paying out or drawing in) is not specified. Similarly, causing the drive shaft 420 of a drive member 400 to rotate may also be referred to herein as “actuating” the drive element 400. Thus, using this nomenclature, a given actuation element 500 may be actuated by actuating a corresponding drive member 500.

As shown in FIGS. 3 and 4, each drive member 400 also comprises a drive input 410 coupled to the drive shaft 420 such that the drive shaft 420 is constrained to rotate with the drive input 410. As noted above, in a mounted state of the instrument 10 to a manipulator, or as otherwise manipulated manually in manually-operated instrument embodiments, the drive input 410 receives driving forces from a drive output of the manipulator, and these driving forces drive rotation of the drive shaft 420 coupled to the drive input 410. (The driving forces may also be referred to as torques, because they induce rotational motion; herein, references to “force” are not limited to forces urging linear motion, and can include torques that urge rotational motion. Moreover, torques can be provided by manually driven inputs in the case of manually-operated instrument embodiments.) As shown in FIG. 4, a distal end portion of each drive shaft 420 is coupled to a drive input 410, which is rotatably coupled to the chassis 311. In addition, a proximal end portion of each drive shaft 420 may be rotatably coupled to a common proximal support 317 via a hole 318 into which a proximal end of the drive shaft 420 is inserted. Thus, each drive shaft 420 has one rotational bearing at a proximal end portion thereof in the form of the proximal support 317 and another rotational bearing at the distal end portion thereof in the form of the chassis 311 (via the drive inputs 410). The proximal support 317 may be coupled with the instrument shaft 100 via bearings 319 that are rotatable around axes that are perpendicular to the longitudinal axis of the shaft 100, which allows the proximal support 317 to translate along the shaft 100 along with the other portions of the transmission assembly 300, including chassis 311 (or allows the shaft 100 to translate along the proximal support 317 in the case where the chassis 311 is held stationary). The bearings 319 also provide lateral support to the proximal support 317 and help to hold the proximal support 317 in its intended position. Additional supports (not illustrated) may also be used to couple the proximal support 317 to the chassis 311.

In addition to the drive input 410 and the drive shaft 420, each drive member 400 also comprises one or more actuation element handling components that couple the drive member 400 to at least one corresponding actuation element 500. The actuation element handling components of each drive member 400 are coupled to the drive shaft 420 thereof such that rotation of the drive shaft 420 causes the actuation element component to actuate the corresponding actuation element(s) 500 coupled thereto. In the case of the insertion drive member 400_1, the actuation element handling component comprises a rotatable drum or capstan 415 (hereinafter drum 415). In the case of the non-insertion drive members 400_2 to 400_5, their respective actuation element handling components comprise actuation transfer mechanisms 430. Specifically, in the embodiment illustrated in FIG. 4, each non-insertion drive member 400_2 to 400_5 comprises two actuation transfer mechanism 430 and 430′ that are coupled to corresponding actuation elements 500 and 500′. The actuation transfer mechanisms 430 may be used as the actuation element handling device 27 described above. These actuation element handling components and their interactions with the actuation elements 500 are described in greater detail below.

### Insertion Drive Member and Corresponding Actuation Elements

With continued reference to FIGS. 4 and 5, an embodiment of the insertion drive member 400_1 will be described in greater detail below. FIG. 5 is a schematic diagram showing how actuation elements 500 are routed and connected to other components of the instrument, such as the drive members 400 of the transmission assembly 300. The diagram in FIG. 5 is not intended to show actual sizes, actual shapes, actual or relative physical locations of components, or the actual paths taken by actuation elements 500, but instead FIG. 5 shows the connections and routing schematically and in simplified form to aid understanding. It should be understood that additional handling components besides those illustrated, such as pulleys, could be interposed in the actuation element paths, and in actual implementations the actuation element paths may deviate from those shown. Moreover, in FIG. 5, the instrument shaft 100 is indicated schematically by dashed lines, and different portions of the shaft 100 are shown with different widths to facilitate illustration of the disposition of the actuation elements 500 relative to the shaft 100 in and along different portions thereof, but it should be understood that the illustrated dimensions of the shaft 100 are not meant to be accurate or to scale.

As noted above, the insertion drive member 400_1 comprises a rotatable drum 415 to actuate (i.e., draw in and pay out) the actuation elements 500 that are coupled to the drum 415. The term “drum” as used herein is intended to refer broadly to any component having a rotational axis and a bearing surface extending generally circumferentially (although not necessarily in a perfect circle) around the rotational axis around which flexible pullable actuation elements 500 (such as cables, wires, filaments, chains, straps, belts, etc.) can be wound by rotation of the drum 415. The drum 415 may also be referred to or function as, for example, a cylinder, a spool, a capstan, a windlass, a winch, or the like. The drum 415 may have guide elements, such as grooves and/or ridges (shown in FIG. 4), to guide actuation elements 500 as they wind around the drum 415. In other embodiments, the drum 415 may omit such guide elements and have a generally smooth surface.

Multiple pullable actuation elements 500 are coupled to and wound around drum 415 such that rotation of the drum 415 draws in or pays out the actuation elements 500, depending on direction of rotation. As shown in FIGS. 4-6, one or more of the actuation elements 500 that are coupled to the drum 415 are also coupled to and actuatable by a non-insertion drive member 400 (e.g., drive members 400_2 to 400_5). But, as will be described in more detail below, these actuation elements 500 can move past or through the non-insertion drive members 400 (e.g., drive members 400_2 to 400_5) to which they are coupled when the actuation elements 500 are actuated by the drum 415 (due to the actuation transfer mechanisms 430, described below); therefore, the non-insertion drive members 400 (e.g., drive members 400_2 to 400_5) can be ignored when considering the operation of the insertion drive member 400_1.

At least one of the actuation elements 500 coupled to the drum 415 (e.g., actuation element 500_1) extends proximally from the transmission assembly 300 and is coupled (directly or indirectly) to a proximal portion 100b of the shaft 100 at a position proximal of the transmission assembly 300, as shown schematically in FIG. 5. At least one other actuation element 500 coupled to the drum 415 (e.g., actuation elements 500_3, 500_3′, 500_4, and 500_4′) extends distally from the transmission assembly 300 and is coupled (directly or indirectly) to a distal portion 100a of the shaft 100 at a position distal of the transmission assembly 300, as shown schematically in FIG. 5. As shown in FIGS. 4 and 5, each actuation element 500 that is coupled to the proximal portion 100b of the instrument shaft 100 (e.g., actuation element 500_1) is wound around the drum 415 in an opposite direction than the actuation elements 500 that are coupled to the distal portion 100a of the shaft 100 (e.g., actuation elements 500_3, 500_3′, 500_4, and 500_4′). Therefore, rotation of the drum 415 in one direction draws in the actuation element(s) 500 that are coupled to the proximal portion 100b (e.g., actuation element 500_1) while simultaneously paying out the actuation elements 500 coupled to the distal portion 100a (e.g., actuation elements 500_3, 500_3′, 500_4, and 500_4′), and rotation of the drum 415 in an opposite direction has the opposite effect. The drawing in of the actuation element(s) 500 coupled to the proximal portion 100b pulls the instrument shaft 100 to move in a distal direction relative to the transmission assembly 300. Conversely, the drawing in of the actuation elements 500 coupled to the distal portion 100a pulls the instrument shaft 100 to move in a proximal direction relative to the transmission assembly 300. Thus, the insertion drive member 400_1 is actuatable to control the insertion degree of freedom of motion of the instrument 10, or in other words to drive relative motion of the shaft 100 and the transmission assembly 300.

As noted above, in the embodiment illustrated in FIGS. 4-7C, the one or more actuation elements 500 that are coupled to the proximal portion 100b of the shaft 100 comprise the actuation element 500_1. In some embodiments, the actuation element 500_1 is not coupled to any of the non-insertion drive members 400. As shown in FIG. 5, the actuation element 500_1 is routed from the drum 415 to a waterfall pulley 530 that has an axis of rotation perpendicular to the axis of rotation of the drum 415. The waterfall pulley 530 redirects the actuation element 500_1 to extend proximally along the shaft 100. The actuation element 500_1 extends along the shaft 100 through a groove 110 (not illustrated) in an outer surface of the shaft 100 (FIG. 4 illustrates another groove 110, which is similar to the one through which the actuation element 500_1 extends). As shown schematically in FIG. 5, the actuation element 500_1 extends proximally until it reaches a coupling location 565_1 at which the actuation element 500_1 is coupled either directly or indirectly to the shaft 100. The coupling location 565_1 may be part of the shaft 100 or part of some other component that is itself coupled (directly or indirectly) to the shaft 100. The coupling location 565_1 may be located somewhere along the proximal portion 100b of the shaft 100, for example at a position that remains proximal of the transmission assembly 300 throughout a range of motion of the shaft 100 and transmission assembly 300 relative to one another. Thus, when the distal insertion actuation element 500_1 is drawn in, it pulls on the shaft 100 and urges the shaft 100 to move in the distal direction.

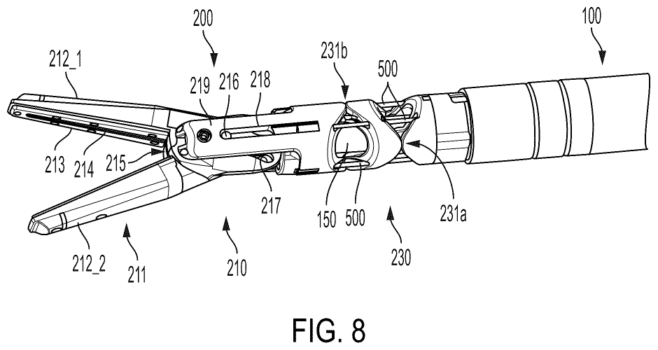

As noted above, in the embodiment illustrated in FIGS. 4-7C, the actuation elements 500 that are coupled to the distal portion 100a of the shaft 100 comprise actuation elements 500_3, 500_3′, 500_4, and 500_4′. In the embodiment of FIGS. 4-7C, all of these actuation elements 500_3, 500_3′, 500_4, and 500_4′ are coupled to both the drum 415 and to one of the non-insertion drive members 400_3 and 400_4. Specifically, as shown in FIG. 5, the actuation elements 500_3, 500_3′, 500_4, and 500_4′ have respective proximal end portions coupled to the drum 415, extend from the drum 415 to couple with corresponding drive members 400_3 and 400_4 via actuation transfer mechanisms 430_3 and 430_4 (described below), extend from the drive members 400_3 and 400_4 to corresponding waterfall pulleys 530, and then from the waterfall pulleys 530 extend distally along and through the shaft 100 to the distal portion 100a of the shaft 100 where respective distal end portions of the actuation elements 500_3, 500_3′, 500_4, and 500_4′ are coupled to coupling points 565_3, 565_3′, 565_4, and 565_4′, respectively. The coupling points 565_3, 565_3′, 565_4, and 565_4′ may be coupled directly or indirectly with the distal portion 100a of the shaft 100. In the embodiment in FIGS. 4-7C, the coupling points 565_3, 565_3′, 565_4, and 565_4′ are part of one or more articulable structures 230, which are coupled to the shaft 100. Specifically, in the embodiment illustrated in FIG. 5, the articulable structure 230 comprises a wrist mechanism comprising two joints comprising a first link 231a pivotably coupled to shaft 100 to pivot along a first direction and a second link pivotably coupled to the first link 231a to pivot along a second direction substantially orthogonal to the first direction. (In FIG. 5, the links 231a and 231b appear to pivot in the same plane due to the simplified schematic nature of the drawing, but in practice they may pivot along different directions, for example as shown in FIG. 8). In this embodiment, a first pair of actuation elements 500_3 and 500_3′ are coupled to coupling points 565_3 and 565_3′ that are part of the first link 231a and a second pair of actuation elements 500_4 and 500_4′ are coupled to coupling points 565_4 and 565_4′ that are part of the second link 231b. Thus, simultaneous actuation of the actuation elements 500_3 and 500_3′ in opposite directions causes first link 231a to pivot relative to the shaft 100 along the first direction, and simultaneously actuation of actuation elements 500_4 and 500_4′ in opposite directions causes second link 231b to pivot relative to the first link 231a along the second direction. In other embodiments, the articulable structure 230 comprises a wrist mechanism similar to that described above, but instead of pairs of the actuation elements 500 being coupled respectively to each link 231a and 231b, in these embodiments all four actuation elements 500_3, 500_3′, 500_4, and 500_4′ are coupled to the second link 231b. In such embodiments, the operation of the wrist mechanism may be the same as that described above with respect to the embodiment of FIG. 5. In other embodiments, some or all of the coupling points 565_3, 565_3′, 565_4, and 565_4′ may be parts of other movable component 200 other than an articulable structure 230, parts of the shaft 100, or parts of some other component of the instrument 10 which is coupled to the shaft 100, such as any moveable components that may utilize a coordinated pair of pull-pull actuation elements to move the moveable component.

In the embodiment of FIG. 5, the coupling points 565_3, 565_3′, 565_4, and 565_4′ and the one or more articulable structures 230 to which they are coupled may be configured such that, when all of the actuation elements 500_3, 500_3′, 500_4, and 500_4′ are simultaneously drawn in at the same rate, then the one or more articulable structures 230 are locked in whatever their current position is by equal and opposite tension applied thereto from opposing pairs of the actuation elements 500_3, 500_3′, 500_4, and 500_4′. Thus, when all of the actuation elements 500_3, 500_3′, 500_4, and 500_4′ are simultaneously drawn in, the one or more articulable structures 230 do not articulate and instead a net force in the proximal direction is applied to the articulable structures 230, and hence also to the shaft 100 to which they are coupled, resulting in proximal translation of the shaft 100. Because the actuation elements 500_3, 500_3′, 500_4, and 500_4′ are all wrapped around the drum 415 in a same direction, rotation of the drum 415 in one direction (e.g., clockwise in FIG. 4) draws in all of the actuation elements 500_3, 500_3′, 500_4, and 500_4′ simultaneously, resulting in proximal translation the shaft 100 along the insertion axis relative to the transmission assembly 300.

Thus, the actuation element 500_1 drives the insertion degree of freedom of motion along one direction when the insertion drive member 400_1 is rotated to draw in the actuation element 500_1, and the actuation elements 500_3, 500_3′, 500_4, and 500_4′ drive the insertion degree of freedom of motion along an opposite direction when the insertion drive member 400_1 is rotated to draw in the actuation elements 500_3, 500_3′, 500_4, and 500_4′.

During actuation of the insertion degree of freedom of motion, the pushable actuation elements 150 and 160 remain stationary relative to the instrument shaft 100. This helps to ensure that actuation of the insertion degree of freedom of motion does not inadvertently cause actuation of movable components that are driven by the actuation elements 150 and 160, such as a component of the end effector 210. The actuation elements 150 and 160 are held stationary relative to the shaft 100 while the shaft 100 is driven to move relative to the transmission assembly 300 because the actuation elements 500 that drive the actuation elements 150 and 160 are configured to cause the actuation elements 150 and 160 to also move relative to the transmission assembly 300 along with the shaft 100 relative to the transmission assembly 300. More specifically, during actuation of the insertion degree of freedom of motion, the actuation elements 150 and 160 are driven to move relative to the transmission assembly 300 in the same direction and for the same distance as the shaft 100, resulting in a net zero motion of the actuation elements 150 and 160 relative to the shaft 100.

The actuation elements 500 coupled to the actuation elements 150 and 160 are configured to drive the above-described motion of the actuation elements 150 and 160 during actuation of the insertion degree of freedom of motion due to the manner in which the actuation elements 500 are coupled to the drum 415 and to the actuation elements 150 and 160. In particular, as shown in FIG. 5, the actuation elements 500_2 and 500_2′ have proximal ends coupled to the drum 415 and distal ends coupled to coupling points 565_2 and 565_2′ on the actuation element 150, with intermediate portions extending from the drum 415 to the drive member 400_2, from the drive member 400_2 to waterfall pulleys 530, and from the waterfall pulleys 530 along and through the shaft 100 to the coupling points 565_2 and 565_2′. Similarly, the actuation elements 500_5 and 500_5′ are coupled to the drum 415 and the coupling points 565_5 and 565_5′. The actuation elements 500_2 and 500_2′ and the coupling points 565_2 and 565_2′ are arranged such that actuation of the actuation elements 500_2 and 500_2′ in opposite directions drives translation of the actuation element 150 relative to the transmission assembly 300. The same is true for the actuation elements 500_5 and 500_5′, the coupling points 565_5 and 565_5′, and the actuation element 160. Moreover, the actuation elements 500_2 and 500_2′ are wrapped around the drum 415 in opposite directions from one another, and therefore when the drum 415 is rotated to drive the insertion degree of freedom of motion, the drum 415 actuates the actuation elements 500_2 and 500_2′ in opposite directions, thus causing the actuation element 150 to move relative to the transmission assembly 300. The same is true for the actuation elements 500_5 and 500_5′ and the actuation element 160. Because such rotation of the drum 415 that causes the translation of the actuation elements 150 and 160 relative to the transmission assembly 300 also causes the shaft 100 to simultaneously move relative to the transmission assembly 300 in the same direction, the actuation elements 150 and 160 remain stationary relative to the shaft 100.

In some embodiments, the transmission assembly 300 may include another actuation element (not illustrated), which is dedicated for driving the proximal translation of the shaft 100 along the insertion axis and not for driving any other movable components 200. For example, such an actuation element may be coupled to the drum 415 and extend distally from the transmission assembly 300 to couple directly to a distal portion 100b of the shaft 100, such that pulling in the actuation element 500 causes proximal translation of the shaft 100.

### Non-Insertion Drive Members and Corresponding Actuation Elements

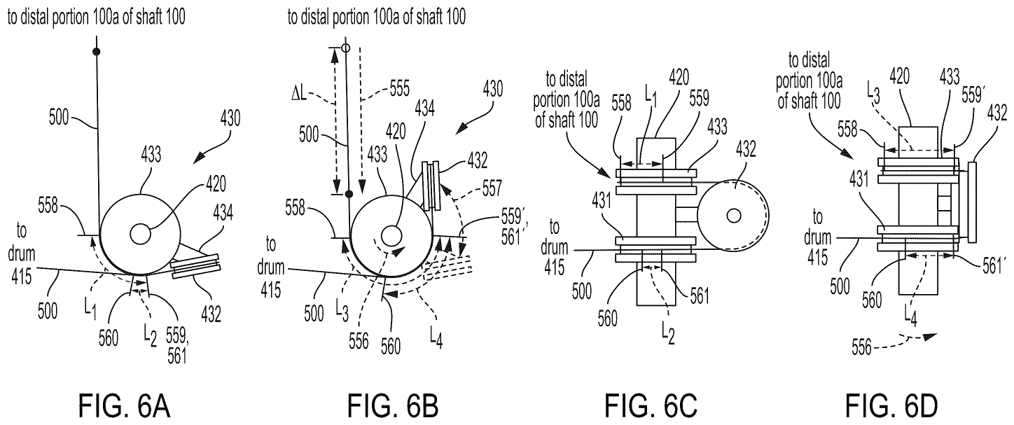

With reference to FIGS. 4-7C, the non-insertion drive members 400 will be described in greater detail below. FIGS. 6A and 6B illustrate plan views of an actuation transfer mechanism 430 (described in greater detail below) in two states from a perspective above (i.e., proximal of) the actuation transfer mechanism 430. FIGS. 6C and 6D illustrate side views of the actuation transfer mechanism 430 in the states of FIGS. 6A and 6B, respectively.

As noted above, at least some of the non-insertion drive members 400 comprise actuation transfer mechanisms 430 to couple with and actuate actuation elements 500, as shown in 4-6D. In FIGS. 4 and 5, all of the non-insertion drive members 400_2 to 400_5 comprise two actuation transfer mechanisms 430. For example, the drive member 400_2 comprises an actuation transfer mechanism 430_2 and an actuation transfer mechanism 430_2′, both coupled to the same drive shaft 420 of the drive member 400_2, and likewise for the other non-insertion drive members 400_3 to 400_5. In other embodiments, some non-insertion drive members 400 might not have an actuation transfer mechanism 430.

An actuation transfer mechanism 430 of a given drive member 400 is configured to allow both the insertion drive member 400_1 and the given non-insertion drive member 400 to actuate the same actuation element 500 independently of one another. In other words, if the drum 415 is held stationary while a given drive member 400 is rotated, the actuation transfer mechanism 430 of the given drive member 400 actuates (pays out or draws in) the corresponding actuation element cable 500. Likewise, if the drum 415 is rotated while the given drive member 400 is held stationary, the actuation transfer mechanism 430 allows the drum 415 to actuate the actuation element 500. Thus, the actuation transfer mechanism 430 can allow a transfer of actuation of the same actuation element 500 between the drum 415 and the corresponding drive member 400 to which the actuation element 500 is coupled.

As shown in FIGS. 4 and 6A-6D, in some embodiments an actuation transfer mechanism 430 comprises a first pulley 431 rotatably coupled to and coaxial with the drive shaft 420, a second pulley 432 radially offset from the drive shaft 420 and having an axis of rotation oriented transverse to an axis of rotation of the drive shaft 420, and a third pulley 433 rotatably coupled to and coaxial with the drive shaft 420. The second pulley 432 is attached to an arm member 434 coupled to and extending generally radially from the drive shaft 420 and constrained to rotate with the drive shaft 420.

The manner in which the actuation elements 500 couple with the actuation transfer mechanisms 430 is described below using the actuation transfer mechanism 430_3 of the drive member 400_3, as shown in FIG. 4, as an example. The same principles apply to the other actuation transfer mechanisms 430 and their respectively corresponding actuation elements 500. As shown in FIG. 4, the actuation element 500_3 is routed from the drum 415 to the first pulley 431 such that the actuation element 500_3 begins to wrap around the first pulley 431 and hence also the drive shaft 420 in a first direction, then the actuation element 500_3 is looped around the second pulley 432 and redirected back to the third pulley 433 to wrap around third pulley 433 and hence the drive shaft 420 again in a second direction opposite the first direction. From the third pulley 433, the actuation element 500_3 extends to a waterfall pulley 530, which redirects the actuation element 500_3 to extend along the shaft 100.

The actuation transfer mechanisms 430 as described above allow the actuation elements 500 coupled thereto to move past or through the non-insertion drive members 400 when actuated by the insertion drive member 400_1. In particular, using the actuation transfer mechanism 430_3 as an example, if the actuation element 500_3 is actuated by the insertion drive member 400_1, the pulleys 431, 432, and 433 of the actuation transfer mechanism 430_3 rotate around their respective axes in response to actuation of the actuation element 500_3, thereby allowing the actuation element 500_3 to be paid out or drawn in through the actuation transfer mechanism 430_3 without actuation (rotation) of the drive member 400_3. Thus, though the drive member 400_3 is held stationary, the actuation element 500_3 can nevertheless be actuated, for example by the insertion drive member 400_1. In other words, the actuation transfer mechanism 430_3 allows the insertion drive member 400_1 to actuate the actuation element 500_3 independently of the drive member 400_3. The same is true of the other actuation transfer mechanisms 430 and their respectively corresponding actuation elements 500. Thus, the actuation transfer mechanisms 430 allow the insertion drive member 400_1 to actuate all of the actuation elements 500 coupled thereto simultaneously to drive the insertion degree of freedom of motion independently of actuation of any of the non-insertion drive members 400_2 to 400_5.

On the other hand, the actuation transfer mechanism 430 as described above also allows the non-insertion drive members 400 to actuate their respectively corresponding actuation elements 500, notwithstanding the fact that the actuation elements 500 are free to move through the actuation transfer mechanisms 430. Returning to the actuation transfer mechanism 430_3 as an example, when the drive shaft 420 of the drive member 400_3 is rotated, the arm 434 of the actuation transfer mechanism 430_3 also is rotated with the shaft 420, which causes the second pulley 432 coupled to the arm 434 to revolve around the drive shaft 420. Because the actuation element 500_3 is looped around the second pulley 432, the revolution of the second pulley 432 around the drive shaft 420 pays out or draws in the actuation element 500_3, depending on the direction of motion. Specifically, revolution of the second pulley 432 around the drive shaft 420 in a one direction (clockwise in the case of the drive member 400_3), increases the amount of the actuation element 500_3 that is wound around the first and third pulleys 431 and 433, thus drawing in the actuation element 500_3. Conversely, revolution of the second pulley 432 around the drive shaft 420 in an opposite direction (counterclockwise in the case of the drive member 400_3) decreases the amount of the actuation element 500_3 that is wound around the first and third pulleys 431 and 433, thus drawing in the actuation element 500_3. Thus, the drive member 400_3 can actuate the actuation element 500_3 even if the insertion drive member 400_1 is held stationary. The same is true of the other actuation transfer mechanisms 430 and their respectively corresponding actuation elements 500. Thus, the actuation transfer mechanisms 430 allow the non-insertion drive members 400_2 to 400_5 to independently actuate their corresponding actuation elements 500, independently of the insertion drive member 400_1.

The manner in which a non-insertion drive member 400 actuates an actuation element 500 via the actuation transfer mechanism 430 can be better understood by considering FIGS. 6A-6D, which illustrate schematically and in greater detail an actuation transfer mechanism 430. FIGS. 6A and 6B illustrate the actuation transfer mechanism 430 in a plan view from a perspective proximal of the actuation transfer mechanism 430, while FIGS. 6C and 6D illustrate a side view of the actuation element 430. In FIGS. 6A and 6C, the actuation transfer mechanism 430 is in a first state, and in FIGS. 6B and 6D the actuation transfer mechanism 430 is in a second state, in which the drive shaft 420 has been rotated counterclockwise (in the view of the figure) relative to the first state, as indicated by the arrow 556. The rotation of the drive shaft 420 causes the second pulley 432 to revolve counterclockwise around the drive shaft 420, as indicated by the arrow 557 in FIGS. 6B and 6D. In the first state shown in FIGS. 6A and 6C, the portion of the actuation element 500 that is wound around the third pulley 433 has a length L₁ from where it initially meets the third pulley 433 (location 558) to where it leaves the third pulley 433 (location 559) to extend to the second pulley 432, and the portion of the actuation element 500 that is wound around the first pulley 431 has a length L₂ from where it initially meets the first pulley 431 (location 560) to where it leaves the first pulley 431 to extend to the second pulley 432 (location 561). In the second state shown in FIGS. 6B and 6D (after rotation of the drive shaft 420), the portion of the actuation element 500 that is wound around the third pulley 433 has a length L₃ from where it initially meets the third pulley 433 (location 558) to where it leaves the third pulley 433 (location 559′) and the portion of the actuation element 500 that is wound around the first pulley 431 has a length L₄ from where it initially meets the first pulley 431 (location 560) to where it leaves the first pulley 431 (location 561′). As can be seen by comparing the lengths L₁ and L₂ illustrated in FIG. 6A with the lengths L₃ and L₄ illustrated in FIG. 6B, the rotation of the drive member 400 about the drive shaft 420 increases the total length of the actuation element 500 that is wound around the first and third pulleys 431 and 433. Specifically, the total length of the actuation element 500 that is wound around the first and third pulleys 431 and 433 in the first state is L₁+L₂, while in the second state it is L₃+L₄, where L₄>L₂ and L₃>L₁. Thus, the total length of the actuation element 500 that is wound around the first and third pulleys 431 and 433 has increased by an amount ΔL=L₃+L₄-(L₁+L₂) between the first and second states. Because one end portion of the actuation element 500 is attached to the drum 415 and thus held stationary, the other end portion of the actuation element 500, which is attached to a distal portion 100a of the shaft 100 (e.g., via a movable component 200), will be drawn in by the distance ΔL to provide for the increased length of the actuation element 500 wrapped round the first and third pulleys 431 and 433. The opposite would occur if the drive shaft 420 were rotated in the opposite direction (clockwise in the view of FIGS. 6A-6B), i.e., the length of the cable 500 wound around the pulleys 431 and 433 would shorten, resulting in the actuation element 500 paying out.

In the illustrated embodiment, the pulleys 431, 432, and 433 are rotatable around their axes of rotation, which may reduce friction and makes actuation of the actuation elements 500 easier. However, in some embodiments one, some, or all of the pulleys 431, 432, and 433 could be replaced with non-rotating bearings. Operation of the actuation transfer mechanism 430 would be the same in such an embodiment, except that the actuation element 500 would slide relative to the bearings rather than the pulleys 431, 432, and 433 rotating. This may result in increased friction, but in some circumstances this may be acceptable.

In the embodiment in FIGS. 4 and 5, the actuation transfer mechanisms 430_3 and 430_3′ have reversed configurations relative to one another, meaning that the actuation transfer mechanisms 430_3 and 430_3′ are configured such that the actuation elements 500_3 and 500_3′ coupled thereto are wound around the drive shaft 420_3 in opposite directions as one another, and therefore rotation of the drive shaft 420_3 in one direction causes the actuation transfer mechanisms 430_3 and 430_3′ to actuate the actuation elements 500_3 and 500_3′, respectively, in opposite directions, i.e., paying out one while drawing in the other, and vice versa. The actuation transfer mechanisms 430_3 and 430_3′ may be arranged with the aforementioned reversed configurations by, for example, positioning the respective second pulleys 432 thereof on opposite sides of their respectively corresponding arms 434 as one another, resulting in opposite winding directions for the actuation elements 500_3 and 500_3′ coupled respectively thereto. In other words, the second pulleys 432 are arranged to face in the same direction as one another when their respective arms 434 extend in opposite directions from one another, as shown in FIG. 4, which is equivalent to the second pulleys 432 facing in opposite directions as one another when their respective arms 434 are aligned in the same direction. In some embodiments, the actuation transfer mechanisms 430_4 and 430_4′ also have reversed configurations relative to one another such that rotation of the drive shaft 420_4 actuated the actuation elements 500_4 and 500_4′ in opposite directions.

As already noted above, in some embodiments, the actuation elements 500_3, 500_3′, 500_4, and 500_4′ are coupled to one or more articulable structures 230, and when all drawn in simultaneously, these actuation elements 500 lock the articulable structures 230 and drive translation of the shaft 100. However, when some of these actuation elements 500_3, 500_3′, 500_4, and 500_4′ are drawn in while others are simultaneously paid out, then the articulable structures 230 are driven to articulate along one or more degrees of freedom of motion, while the shaft 100 is held stationary.

For example, in the embodiment illustrated in FIG. 5, a first articulable structure 230_1 is coupled to actuation elements 500_3 and 500_3′ such that simultaneous actuation of the actuation elements 500_3 and 500_3′ in opposite directions drives movement of the first articulable structure 230_1 along a degree of freedom of motion (e.g., yaw). Thus, in this embodiment, actuation of the drive member 400_3 can drive motion of the articulable structure 230_1 along a degree of freedom of motion, because actuation of the drive member 400_3 results in simultaneous actuation of the actuation elements 500_3 and 500_3′ in opposite directions, as described above. Similarly, in this embodiment, the second articulable structure 230_2 is coupled to the actuation elements 500_4 and 500_4′ such that simultaneous actuation of the actuation elements 500_4 and 500_4′ in opposite directions drives movement of the second articulable structure 230_2 along a degree of freedom of motion (e.g., pitch). Thus, in this embodiment, actuation of the drive member 400_4 can drive motion of the articulable structure 230_2 along a degree of freedom of motion, because actuation of the drive member 400_4 results in simultaneous actuation of the actuation elements 500_4 and 500_4′ in opposite directions, as described above.

In other movements, instead of each drive member 400_3 and 400_4 driving separate degrees of freedom of motion the articulable structures 230, the actuation elements 500_3, 500_3′, 500_4, and 500_4′ may be coupled to the articulable structures 230 such that coordinated actuation of both drive member 400_3 and 400_4 is needed to actuate either degree of freedom of motion. For example, instead of having two pairs of actuation elements 500 each coupled to a different articulable structure 230 having a single degree of freedom of motion, with the actuation elements 500 of a pair being coupled to opposite sides of their respective articulable structure 230, in some embodiments all four actuation elements 500_3, 500_3′, 500_4, and 500_4′ may be coupled to the same articulable structure 230 which has multiple degrees of freedom of motion. In such an arrangement, different types of motion of the articulable structure 230 may be obtained based on the combination of which of the actuation elements 500_3, 500_3′, 500_4, and 500_4′ are drawn in and which are paid out.

The actuation transfer mechanism 430_2 and 430_2′ are similarly arranged with reversed configurations relative to one another so as to actuate the actuation elements 500_2 and 500_2′ in opposite directions. As noted above, the actuation elements 500_2 and 500_2′ are coupled to the pushable actuation element 150 such that, when actuated in opposite directions, they drive translation of the pushable actuation element 150 relative to the transmission assembly 300. More specifically, as shown schematically in FIG. 5 and in perspective in FIGS. 7A and 7C, the actuation element 500_2 extends distally from the transmission assembly 300 to couple with the actuation element 150 at a coupling point 565_2 that is distal of the transmission assembly 300. Thus, when the actuation element 500_2 is drawn in, the actuation element 500_2 pulls the actuation element 150 in a proximal direction relative to the transmission assembly 300. Conversely, as shown schematically in FIG. 5 and in perspective in FIGS. 7A and 7B, the actuation element 500_2′ extends distally from the transmission assembly to couple with the actuation element 150 at a coupling point 565_2′ that is proximal of the transmission assembly 300. Thus, when the actuation element 500_2′ is drawn in, the actuation element 500_2′ pulls the actuation element 150 in a distal direction relative to the transmission assembly 300. Thus, actuation of the drive member 400_2 results in simultaneous actuation of the actuation elements 500_2 and 500_2′ in opposite directions, which drives translation of the actuation element 150 relative to the shaft 100. The direction of translation of the actuation element 150 will depend on the direction of rotation of the shaft 420 of the drive member 400_2.

Similarly, the actuation transfer mechanisms 430 of the drive member 400_5 (not visible in the figures) are arranged with reversed configurations relative to one another so as to actuate the actuation elements 500_5 and 500_5′ in opposite directions. As noted above, the actuation elements 500_5 and 500_5′ are coupled to the pushable actuation element 160 such that, when actuated in opposite directions, they drive translation of the pushable actuation element 160 relative to the transmission assembly 300. More specifically, as shown schematically in FIG. 5 and in perspective in FIGS. 7A and 7B, the actuation element 500_5 extends proximally from the transmission assembly 300 to couple with the actuation element 160 at a coupling point 565_5 that is proximal of the transmission assembly 300. Thus, when the actuation element 500_5 is drawn in, the actuation element 500_5 pulls the actuation element 160 in a distal direction relative to the transmission assembly 300. The actuation element 500_5′ also extends proximally from the transmission assembly 300 to couple with the actuation element 150 at a coupling point 565_5′ that is proximal of the transmission assembly 300, but the actuation element 500_5′ is redirected by a pully 535 coupled to the proximal portion 100b of the instrument shaft 100 such that, when the actuation element 500_5′ is drawn in, the actuation element 500_5′ pulls the actuation element 160 in a proximal direction relative to the transmission assembly 300. Thus, actuation of the drive member 400_5 results in simultaneous actuation of the actuation elements 500_5 and 500_5′ in opposite directions, which drives translation of the actuation element 160 relative to the shaft 100. The direction of translation of the actuation element 160 will depend on the direction of rotation of the shaft 420 of the drive member 400_5.

In FIGS. 5 and 7A-7C, one routing and coupling arrangement of the actuation elements 500_2, 500_2′, 500_5, and 500_5′ relative to the actuation elements 150 and 160 is illustrated, but it should be understood that the same operational effect could be achieved by routing the actuation elements 500_2, 500_2′, 500_5, and 500_5′ differently and/or coupling the actuation elements 500_2, 500_2′, 500_5, and 500_5′ to the actuation elements 150 and 160 differently. For example, in FIGS. 5 and 7A-7C, the actuation elements 500_2 and 500_2′ extend from the transmission assembly 300 in opposite directions and to couple with coupling points 565_2 and 565_2′ located at proximal and distal end portions of the actuation elements 150, but in other embodiments these coupling points 565_2 and 565_2′ may be moved and the actuation elements 500_2 and 500_2′ may extend from the transmission assembly in the same direction. In some embodiments, both coupling points 565_2 and 565_2′ are located distal of the transmission assembly 300, in which case the actuation element 500_2′ is routed distally (instead of proximally) to a pulley located in the shaft 100 at a position distal of the transmission assembly 300, which redirects the actuation element 500_2′ to extend proximally to the coupling point 565_2′. Conversely, in other embodiments both coupling points 565_2 and 565_2′ are located proximally of the transmission assembly 300, in which case the actuation element 500_2 is routed proximally (instead of distally) to a pulley located in the shaft 100 at a position proximal of the transmission assembly 300, which redirects the actuation element 500_2 to extend distally to the coupling point 565_2. As another example, in FIGS. 5 and 7A-7C, the actuation elements 500_5 and 500_5′ extend from the transmission assembly 300 proximally to couple with coupling points 565_5 and 565_5′, but in other embodiments one or both of the actuation elements 500_5 and 500_5′ may extend from the transmission assembly 300 in a distal direction to a pulley that redirects the actuation element 500_5 or 500_5′, as the case may be, to extend in the proximal direction.

As shown in FIGS. 7A-7C, in some embodiments at least a portion of the pushable actuation element 150 is formed from a relatively rigid push element in the form of a tube with a central passage, and the actuation element 160 may extend through the central passage of the actuation element 150. Moreover, in some embodiments, the actuation element 150 may comprise two portions 150a and 150b, as shown in FIG. 5, with the portion 150a located proximal of the articulable structures 230 and the portion 150b extending through the articulable structures 230. The portion 150a of the actuation element 150 may comprise the aforementioned rigid push element, while the portion 150b may comprise a more flexible push element that can flex laterally in one or more directions, such as a push-coil, cut tube, or the like. In particular, in some embodiments the actuation element 150 may comprise a tube that has been cut along the portion 150b to form a flexible cut tube in the portion 150b with the remainder of the tube being uncut and forming the more rigid portion 150a.

In some embodiments, as shown in FIGS. 7A and 7B, the pushable actuation element 160 comprises two portions 160a and 160b. The portion 160a may be a portion to which the actuation elements 500_5 and 500_5′ are coupled. The portion 160a is coupled to a portion 160b, which extends through the central channel of the actuation element 150. In some embodiments, portion 160a may be relatively rigid, which facilitates driving of the pushable actuation element 160 by the actuation elements 500_5 and 500_5′. In some embodiments, a portion of the actuation element 160 may be relatively flexible in lateral directions. For example, in some embodiments, a flexible portion of the actuation element 160 may comprise a pullable actuation element, such as a cable for example, which is supported laterally by the actuation element 150 and thus is capable of also transmitting pushing loads while still being bendable laterally. In other embodiments, the flexible portion of the actuation element 160 may comprise a semi-flexible push member, such as a beam that is relatively flexible along one dimension (e.g., a thinnest dimension of the beam). In some embodiments, the flexible portion of actuation element 160 may comprise at least a portion that extends through the articulable structures 230. In some embodiments, the flexible portion of the actuation element 160 may comprise all of the portion 160a. In other embodiments, the flexible portion of the actuation element 160 may comprise only a distal part of the portion 160a, with a proximal part of the portion 160a being formed from a more rigid member such a bar or tube.

In FIGS. 5, 7A and 7B, the portion 160a is relatively elongated, which may facilitate smoother actuation of the actuation element 150 by reducing the tendency of the portion 160a to cant when pulled (due to the coupling points 565_5 and 565_5′ being offset from the center axis). However, in other embodiments, the portion 160a could be sized or shaped differently, or may even be omitted (in which case the actuation elements 500_5 and 500_5′ may couple directly to the portion 160b). Guide tracks (not illustrated) and/or other stabilization elements may also be provided in the shaft 100 to help guide the motion of the actuation elements 150 and 160 and to prevent canting or other binding issues. In FIGS. 5, 7A and 7B, the coupling points 565_5 and 565_5′ are spaced apart from one another at opposite ends of the portion 160a, but in other embodiments coupling points 565_5 and 565_5′ could be closer together or even at the same general location along the longitudinal dimension of the shaft 100.

The embodiment of FIGS. 2-7C is described above as one non-limiting example of how the drive members 400 may be arranged to interact with the pullable actuation elements 500 and pushable actuation elements 150 and 160, but other embodiments are contemplated herein. Specifically, although five drive members 400 are illustrated in FIGS. 2-7C, other embodiments have more or fewer drive members 400, including any number of drive members 400_1 to 400_N where N is any integer equal to or greater than 2. In addition, although FIGS. 2-7C illustrates five actuation elements 500 with four extending proximally and five extending distally, in other embodiments more or fewer actuation elements 500 may be provided that extend in the proximal direction and more or fewer actuation elements 500 may be provided that extend in the distal direction. Moreover, although FIGS. 2-7C illustrate all of the actuation elements 500 that are driven by one of the non-insertion drive members 400_2 to 400_5 as also being coupled to the insertion drive member 400_1, in other embodiments one or more of the actuation elements 500 may be coupled to a non-insertion drive member 400 without also being coupled to the insertion drive member 400_1. In addition, although FIG. 6 illustrates the actuation elements 500_3, 500_3′, 500_4, and 500_4′ as being coupled to an articulable structure 230, in other embodiments one or more actuation elements 500_3, 500_3′, 500_4, and 500_4′ could be coupled to something other than such an articulable structure 230, such as another type of movable component 200 (e.g., a movable component of an end effector 210), another actuation element (e.g., push-pull rod), or another component of the instrument 10. Furthermore, although FIG. 2-7C illustrate all of the actuation elements 500 that extend distally as being coupled to a non-insertion drive member 400, in some embodiments one or more actuation elements 500 that extend distally are coupled to the insertion drive member 400_1 without being coupled to another non-insertion drive member 400.

### Bearing Mechanisms for Routing Actuation Elements

With continued reference to FIG. 4, an embodiment of a bearing mechanism 440, such as those mentioned above, is described below in greater detail. As described above, at least one of the non-insertion drive members 400 is provided with one or more bearing mechanisms 440 coupled to the drive shaft 420 of the drive member 400. Each bearing mechanism 440 is configured to redirect an actuation element 500 that is actuatable by another drive member 400 as that actuation element 500 extends between the insertion drive member 400_1 and a non-insertion drive member 400. Specifically, in the embodiment shown in FIG. 4, the non-insertion drive member 400_2 is provided with two bearing mechanisms 440 and 440′, which redirect the actuation elements 500_3 and 500_3′, respectively, as they extend between the drum 415 and the drive member 400_3 to which the actuation elements 500_3 and 500_3′ are coupled for actuation. The bearing mechanisms 440 and 440′ may be positioned on the drive shaft 420 at locations along the proximal-distal direction that generally align with the take-up points of the actuation transfer mechanisms 430 and 430′ of the drive member 400_3, such that the bearing mechanisms 440 and 440′ can redirect the actuation elements 500_3 and 500_3′. Similarly, the drive member 400_5 is provided with two bearing mechanisms 440 and 440′ (not visible in FIG. 4), which redirects the actuation element 500_4 and 500_4′ as they extends between the drum 415 and the drive member 400_4. The bearing mechanisms 400 may have a generally cylindrical portion with an outward facing surface thereof acting as a bearing surface that engages with and redirects the actuation element 500.

In some embodiments, the bearing mechanisms 440 may be rotatably coupled to the respective drive shafts 420. Thus, the bearing mechanisms 440 may act as a pulley, rotating as the actuation element 500 engaged therewith moves past the drive member 400, such as when the actuation element 500 is actuated by the drum 415. The bearing mechanism 440 may comprise an internal bearing (not shown) that rotatably couples the bearing mechanism 440 to the drive shaft 420. In some embodiments, the internal bearing may comprise a low friction rotatable bearing, such as a ball bearing, deep-groove ball bearings, angular contact bearings, cylindrical roller bearings, thrust bearings, tapered roller bearings, etc., to reduce the amount of friction resisting rotation of the bearing mechanism 440. In other embodiments, the internal bearing may comprise a plain bearing. In some embodiments, grease, oil, or other lubricants may be used with the internal bearing reduce friction and wear. Material combinations of the internal bearing and the drive shaft 420, or surface treatments applied thereon, may also be chosen to reduce friction, wear, or galling. For example, relative low friction (lubricious) materials such as PTFE, UHMW, Acetal, brass or bronze, Nitronic 60, or other similar materials may be used for a body of the bearing mechanism 440 and/or the drive shaft 420 to reduce the amount of friction resisting rotation of the bearing mechanism 440.

Because the bearing mechanisms 440 are provided on the non-insertion drive members 400_2 and 400_5, the drive members 400_2 and 400_5 can be positioned at locations of the transmission assembly 300 that might otherwise not be feasible. For example, as shown in FIG. 4, the drive member 400_2 is positioned at a location between the drive member 400_3 and the drive member 400_1 at which the drive member 400_2 would have otherwise interfered with the actuation element 500_3 as it extended between the drum 415 and the drive member 400_3, but because the bearing mechanism 440 is provided on the drive member 400_2 the actuation element 500_3 is redirected around the drive member 400_2. Similarly, the drive member 400_5 can be positioned between the drive member 400_4 and the drive member 400_1 due to the bearing mechanism 440. This ability to position the drive members 400 relative to one another at locations that would otherwise not be feasible can allow the transmission assembly 300 to be made smaller, or allow more free space in which additional components could be included at a given size/footprint of transmission assembly 300.

### End Effector and Articulable Structures

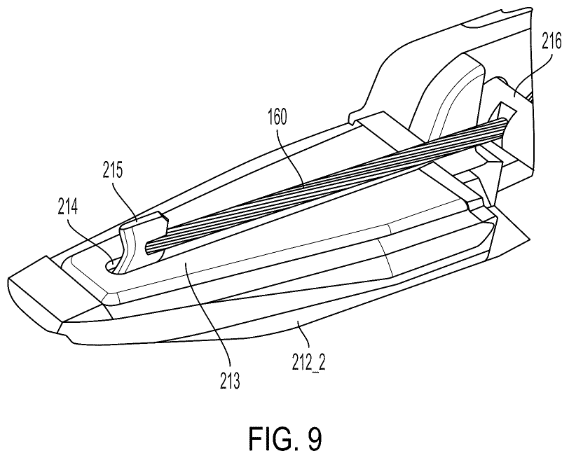

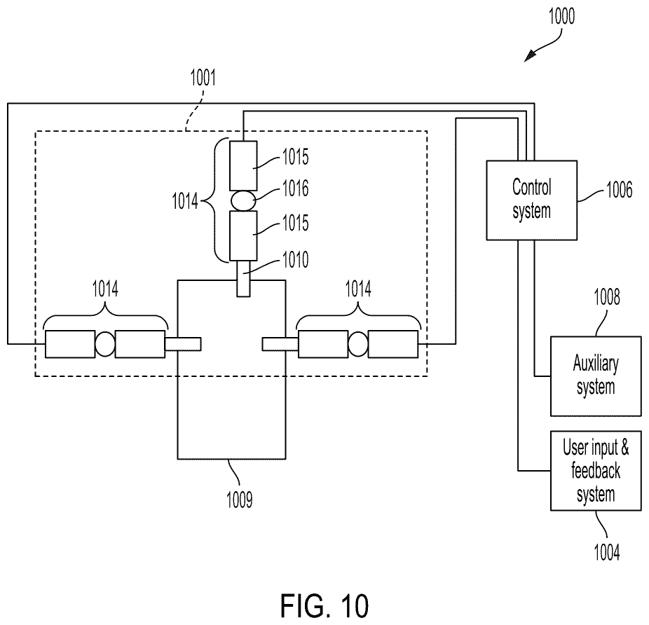

Turning now to FIGS. 8 and 9, embodiments of an end effector 210 and articulable structures 230 are described. The end effector 210 may be coupled to a distal end portion of the shaft 100, for example via the articulable structures 230.

In the embodiment of FIGS. 8 and 9, the end effector 210 is configured as a sealer that grasps, seals, and cuts an object (e.g., tissue). Such a sealer is often employed to cut and seal vessels (e.g., blood vessels) and thus may be referred to as a vessel sealer, but it should be understood that this nomenclature is not limiting and that the sealer can be used on other types of material, including other tissues or non-tissue material.

The end effector 210 comprises a jaw mechanism 211, which has two opposing jaw members 212_1 and 212_2 that are pivotally coupled to a clevis 219 so as to pivot between open and closed configurations. In the open configuration, distal ends of the jaw members 212_1 and 212_2 are spaced apart from one another, while in the closed configuration the distal ends of the jaw members 212_1 and 212_2 are relatively close to one another such that an angle between the jaw members 212_1 and 212_2 is relatively small (zero, in some embodiments). The jaw members 212_1 and 212_2 are driven to move between the open and closed configurations by translation of an actuation link 216, which is coupled with the jaw members 212_1 and 212_2 via ramped slots 217 in the jaw members 212_1 and 212_2 and coupled with the clevis 219 via guide slot 218. The jaw members 212_1 and 212_2 also comprise one or more electrodes 213, which are electrically coupled to electrical energy transport lines (not illustrated). During usage, the electrical energy transport lines may be coupled to an electrical energy sources, such as an electrosurgical unit (ESU), to supply electricity to the one or more electrode 213 to perform an electrosurgical function. In one embodiment, each jaw member 212_1 and 212_2 comprises a bipolar electrode configured to perform bipolar electrosurgical functions, such as bipolar electrosurgical sealing, a material grasped between the jaw members 212_1 and 212_2.

The end effector 210 may also comprise a translatable component 215. The translatable component 215 may be, for example, a cutting element. The translatable component 215 element may be driven to translate between a proximal or home position, illustrated in FIG. 8, and a distal or extended position, illustrated in FIG. 9 (in FIG. 9 the jaw member 212_1 is omitted to improve clarity). In some embodiments, the electrodes 213 may perform a sealing operation on material (e.g., a vessel) grasped between the jaw members 212_1 and 212_2, and thereafter the translatable component 215 element may be fired (driven from the home position to the extended position) while the jaw members 212_1 and 212_2 are still closed and grasping the material such that the translatable component 215 cuts the now sealed material. As shown in FIGS. 8 and 9, the jaw members 212_1 and 212_2 may comprise a track 214 that guides the translatable component 215 as it translated relative to the jaw members 212_1 and 212_2.

The translatable component 215 is driven to move between the proximal and distal positions by translation of the actuation element 160. As shown in FIG. 9, the translatable component 215 is coupled to a distal end portion of the actuation element 160. The actuation element 160 may extend through an opening in the actuation link 216, such that the actuation element 160 can translate relative to the actuation link 216 within a certain range of motion. As noted above, the pushable actuation element 160 can transfer both pushing forces and pulling forces, and thus is able to drive both proximal and distal translation of the translatable component 215 relative to the jaw mechanism 211. In particular, the drive member 400_5 may be actuated to draw in the actuation element 500_5 while paying out the actuation element 500_5′, thus driving the actuation element 160, and hence the translatable component 215 coupled thereto, to move distally. Actuation of the drive member 400_5 in the opposite direction results in the opposite effect, namely proximal translation for the translatable component 215.

As noted above, the jaw mechanism 211 is moved between the open and closed configuration by translation of the actuation link 216. The actuation link 216 may be coupled to and driven by the pushable actuation element 150. For example, in some embodiments the pushable actuation element 150 is configured to push the actuation link 216 in a distal direction, thus closing the jaw mechanism 211. The pushable actuation element 150 may be positioned adjacent to a proximal side of the actuation link 216, such that when the actuation element 150 is driven distally (by the drive member 400_2), a distal end of the actuation element 150 pushes against the actuation link 216 and forces it to move distally. In some embodiments, the actuation element 150 is also coupled to the actuation link 216 such that when the actuation element 150 is driven to translate in the proximal direction (by the drive member 400_2), the actuation element 150 pulls the action link 216 proximally and causes the jaw mechanism 211 to open. In some embodiments, the pushable actuation element 150 may comprise a portion 150b that is configured as a laterally flexible push member as described above, such as a coil pipe or cut tube. In some embodiments, the flexible portion 150b is coupled with the actuation link 216, while in other embodiments the flexible portion 150b extends through the articulable structure 230 and then couples to a more rigid portion (not illustrated) that couples with the articulable structure 230. In other embodiments, the opening of the jaw mechanism 211 may be driven by the actuation element 160 (or by both the actuation elements 150 and 160) being pulled in the proximal direction (by the drive member 400_5).

As shown in FIG. 8, in some embodiments, the articulable structures 230 comprises a wrist mechanism having at least two orthogonal degrees of freedom of motion (e.g., pitch and yaw) provided by two orthogonal joints formed from two links 231a and 231b pivotably coupled together. These joints 231a and 231b are coupled to and driven by actuation elements 500, specifically the actuation elements 500_3, 500_3′, 500_4, and 500_4′ as described above.

The end effector 210 illustrated in FIGS. 8 and 9 is configured as a vessel sealer, but this is just one embodiment of an end effector that can be used in the instrument 10. One of ordinary skill in the art would understand that any other type of end effector could be used with the instrument 10 in lieu of the end effector 210, such as, for example, an end effector configured as a stapler, an electrosurgical device (bipolar or monopolar), an electrocautery devices, an endoscope, forceps, or any other type of end effector. Similarly, any number of articulable structures of any type may be used in addition to or in lieu of the articulable structures 230, including having zero articulable structures in some embodiments.

### Manipulator System

FIG. 10 is a schematic block diagram of an embodiment of a computer-assisted instrument control system 1000 for remote control of instruments in accordance with various embodiments, also referred to herein as a teleoperable instrument system. Such a system can be a medical system that employs robotic technology, as those having ordinary skill in the art are familiar with. The system 1000 comprises a manipulator assembly 1001, a control system 1006, and a user input and feedback system 1004. The system 1000 may also include an auxiliary system 1008 to provide various supporting functionality to the instruments or the overall system. These components of the system 1000 are described in greater detail blow.

The manipulator assembly 1001 comprises one or more manipulators 1014. FIG. 10 illustrates three manipulators 1014, but any number of manipulators 1014 may be included. While a manipulator may comprise a single mechanical link, in the embodiment of FIG. 10, each manipulator 1014 comprises a kinematic structure of two or more links 1015 coupled together by one or more joints 1016. The joints 1016 may impart various degrees of freedom of movement to the manipulator 1014, allowing the manipulator 1014 to be moved around a workspace 1009. For example, some joints 1016 may provide for rotation of links 1015 relative to one another, other joints 1016 may provide for translation of links 1015 relative to one another, and some may provide for both rotation and translation. Some or all of the joints 1016 may be powered joints, meaning a powered drive element may control movement of the joint 1016 through the supply of motive power. Such powered drive elements may comprise, for example, electric motors, pneumatic or hydraulic actuators, etc. Additional joints 1016 may be unpowered joints. FIG. 2 illustrates each manipulator 1014 as having two links 1015 and one joint 1016, but in practice a manipulator may include more links 1015 and more joints 1016, depending on the needs of the system 1000. The more links 1015 and joints 1016 are included, the greater the degrees of freedom of movement of the manipulator 1014.

Each manipulator 1014 may be configured to support and operate one or more instruments 1010. The instruments 1010 may include various types of instruments, including for example industrial instruments and medical instruments (e.g., surgical instruments, imaging instruments, diagnostic instruments, therapeutic instruments, etc.). For example, the instruments 1 and 10 described above may be used as any of the instruments 1010. A manipulator 1014 may comprise an instrument manipulator interface to which an instrument 1010 can be removably coupled. The instrument manipulator interface may be located, for example, at a distal end portion of the manipulator 1014. The instrument manipulator interface may include drive outputs to provide driving forces to drive inputs of the instrument 1010 to control operations of the instrument 1010, such as moving an end-effector of the instrument, opening/closing jaws, driving translating and/or rotating components, etc. The drive outputs may be driven by actuators (e.g., electrical motors, hydraulic actuators, pneumatic actuators, etc.) and may interface with and mechanically transfer driving forces to corresponding drive inputs of the instrument 1010 (directly, or via intermediate drive outputs, which may be part of a sterile instrument adaptor (ISA) (not illustrated)). The ISA may be placed between the instrument 1010 and the instrument interface to maintain sterile separation between the instrument 1010 and the manipulator 1014. The instrument interface may also comprise other interface components (not illustrated), such as electrical interfaces to provide and/or receive electrical signals to/from the instrument 1010. In some embodiments, the manipulator assembly can include flux delivery transmission capability as well, such as, for example, to supply electricity, fluid, vacuum pressure, light, electromagnetic radiation, etc. to the end effector. In other embodiments, such flux delivery transmission may be provided to an instrument through another auxiliary system, described further below.

An example embodiment of an instrument manipulating portion 60 of a manipulator 1014, is illustrated in FIG. 10. The instrument manipulating portion 60 is configured to support and operate an instrument 10 mounted thereon. As shown in FIG. 11, the instrument manipulating portion 60 comprises a base 77 and an instrument holder assembly 70. The base is coupled with a link 1015 of a manipulator 1014, for example by a joint 1016. The instrument holder assembly 70 comprises an outer housing 72 coupled to the base 77 and an instrument holder 71 coupled to the outer housing 72. The instrument holder 71 comprises an interface 73 and an inner portion (not illustrated) that is received within the outer housing 72. The instrument holder 71 may be rotatable relative to the outer housing 72, thus imparting a roll degree of freedom of motion to an instrument 10 mounted thereon.

As shown in FIG. 11, the instrument holder 71 comprises an interface 73 configured to receive and interface with the chassis 311 of the instrument 10 in a mounted state. The interface 73 comprises drive outputs 75 configured to interface with the drive inputs of the instrument, such as drive inputs 410 of the instrument 10, in a mounted state of the instrument to the manipulator. The drive outputs 75 may interface directly with the drive inputs 410, or the drive outputs 75 may interface indirectly with the drive inputs via intermediate drive outputs of an ISA (i.e., the drive outputs 75 engage corresponding intermediate drive outputs of the ISA, and the intermediate drive outputs of the ISA engage corresponding drive inputs 410). The drive outputs 75 may have a shape that is complementary to a shape of the drive inputs 410 and/or a shape of the intermediate drive outputs to allow for mating engagement such that rotation of the drive outputs 75 drives rotation drive inputs 410 and/or intermediate drive outputs. The inner portion of the instrument holder 71 may comprises actuators (not illustrated) coupled to the drive outputs 75 to supply torque to the drive outputs 75. Motive power, such as electricity or pressurized hydraulic or pneumatic fluid, may be provided to the actuators via power supply lines, which may be routed through the base 77 and into the housing 72.

The instrument holder assembly 70 of the embodiment of FIG. 11 comprises a passage 76 extending through the instrument interface 73 and the outer housing 72. In FIG. 11, the passage 76 comprises a generally cylindrical bore, but in other embodiments the passage 76 may have other shapes, such as a slot with a U-shaped cross-section that is open along a lateral side of the instrument holder assembly 70 in addition to being open at opposite axial ends of the instrument holder assembly 70. A portion of the shaft 100 and the sleeve 312 are received in the passage 76 in the mounted state. As described above, the sleeve 312 comprises alignment features 316a and 316b, and the inner surface of the passage 76 may comprise complementary alignment features (not illustrated) to engage with the alignment features 316a and 316b so as to progressively align the instrument 10 as the sleeve 312 is inserted farther into the passage 76.

As shown in FIG. 11, a cannula 74 may be coupled to (or may be an integral part of) the instrument manipulating portion 60. The cannula 74 may be configured for insertion into a patient through an incision or natural orifice so as to allow the shaft 100 of the instrument to be inserted and advanced therethrough. As noted above, a distal end portion of the sleeve 312 of an instrument 10 may extend partially into the cannula 74, and a seal may be provided between the cannula 74 and the exterior of the sleeve 312.

The system 1000 can also include a user input and feedback system 1004 operably coupled to the control system 1006. The user input and feedback system 1004 comprises one or more input devices to receive input control commands to control operations of the manipulator assembly 1001. Such input devices may include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device may be provided with the same degrees of freedom as the associated instrument that they control, and as the input device is actuated, the instrument, through drive inputs from the manipulator assembly, is controlled to follow or mimic the movement of the input device, which may provide the user a sense of directly controlling the instrument. Telepresence input devices may provide the operator with telepresence, meaning the perception that the input devices are integral with the instrument. The user input and feedback system 1004 may also include feedback devices, such as a display device (not shown) to display images (e.g., images of the workspace 1009 as captured by one of the instruments 1010), haptic feedback devices, audio feedback devices, other graphical user interface forms of feedback, etc.

The control system 1006 may control operations of the system 1000. In particular, the control system 1006 may send control signals (e.g., electrical signals) to the manipulator assembly 1001 to control movement of the joints 1016 and to control operations of the instruments 1010 (e.g., through drive interfaces at the manipulators 1014). In some embodiments, the control system 1006 may also control some or all operations of the user input and feedback system 1004, the auxiliary system 1008, or other parts of the system 1000. The control system 1006 may include an electronic controller to control and/or assist a user in controlling operations of the manipulator assembly 1001. The electronic controller comprises processing circuitry configured with logic for performing the various operations. The logic of the processing circuitry may comprise dedicated hardware to perform various operations, software (machine readable and/or processor executable instructions) to perform various operations, or any combination thereof. In examples in which the logic comprises software, the processing circuitry may include a processor to execute the software instructions and a memory device that stores the software. The processor may comprise one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In examples in which the processing circuitry includes dedicated hardware, in addition to or in lieu of the processor, the dedicated hardware may include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry may also include any combination of dedicated hardware and processor plus software.

As noted above, differing degrees of user control versus autonomous control may be utilized in the system 1000, and embodiments disclosed herein may encompass fully user-controlled systems, fully autonomously-controlled systems, and systems having any combination of user and autonomous control. For operations that are user-controlled, the control system 1006 generates control signals in response to receiving a corresponding user input command via the user input and feedback system 1004. For operations that are autonomously controlled, the control system 1006 may execute pre-programmed logic (e.g., a software program) and may determine and send control commands based on the programming (e.g., in response to a detected state or stimulus specified in the programming). In some systems, some operations may be user controlled and others autonomously controlled. Moreover, some operations may be partially user controlled and partially autonomously controlled—for example, a user input command may initiate performance of a sequence of events, and then the control system 1006 may perform various operations associated with that sequence without needing further user input.

While the control system 1006 is illustrated as a separate element in FIG. 11, those having ordinary skill in the art would understand that the control system 1006 may be a distributed system, or portions thereof may be, among one or more of the auxiliary system 1008, the user input and feedback system 1004, and the manipulator system 1001.

The auxiliary system 1008 may comprise various auxiliary devices that may be used in operation of the system 1000. For example, the auxiliary system 1008 may include power supply units, auxiliary function units (e.g., functions such as irrigation, evacuation, energy supply, illumination, sensors, imaging, etc.). As one example, in a system 1000 for use in a medical procedure context, the auxiliary system 1008 may comprise a display device for use by medical staff assisting a procedure, while the user operating the input devices may utilize a separate display device that is part of the user input and feedback system 1004. As another example, in a system 1000 for use in a medical context, the auxiliary system 1008 may comprise flux supply units that provide surgical flux (e.g., electrical power) to instruments 1010. An auxiliary system 1008 as used herein may thus encompass a variety of components and does not need to be provided as an integral unit.

The embodiments described herein (including the instrument 10 and system 1000 described above) may be well suited for use in medical applications. In particular, some embodiments are suitable for use in, for example, surgical, teleoperated surgical, diagnostic, therapeutic, and/or biopsy procedures. Such procedures could be performed, for example, on human patients, animal patients, human cadavers, animal cadavers, and portions or human or animal anatomy. Some embodiments may also be suitable for use in, for example, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments may also be used for benchtop procedures on non-living material and forms that are not part of a human or animal anatomy. Moreover, some embodiments are also suitable for use in non-medical applications, such as industrial robotic uses, and sensing, inspecting, and/or manipulating non-tissue work pieces. In non-limiting embodiments, the techniques, methods, and devices described herein may be used in, or may be part of, a computer-assisted medical system employing robotic technology such as the da Vinci® Surgical Systems commercialized by Intuitive Surgical, Inc., of Sunnyvale, California. Those skilled in the art will understand, however, that aspects disclosed herein may be embodied and implemented in various ways and systems, including manually operated instruments and computer-assisted, teleoperated systems, in both medical and non-medical applications. Reference to the daVinci® Surgical Systems are illustrative and not to be considered as limiting the scope of the disclosure herein.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes may be made without departing from the spirit and scope of this description and the claims. In some instances, well-known circuits, structures, and techniques have not been shown or described in detail in order not to obscure the embodiments. Like numbers in two or more figures represent the same or similar elements.

In the description above, reference is made to “paying out” or “drawing in” actuation elements. As noted above, paying out or drawing in an actuation element refers to increasing or decreasing the length of a segment of the actuation element that extends between a take-off point on the drive member to which the cable is coupled and a coupling point at which the actuation element is operably coupled to the structure whose motion the actuation element drives. The take-off point on a drive member refers to the point at which the actuation element ceases to contact the drive member (or begins to contact the drive member, depending on the point of view). The take-off point of the drive member may move as the drive member is operated—for example, as a capstan is rotated and a actuation element is wound onto or wound off from the capstan, the take-off point on the capstan may move axially along the capstan. As noted above, the coupling point is a point at which the actuation element is operably coupled to the structure that the actuation element is configured to drive, such as a point at which the actuation element is coupled to a movable component (if the actuation element is to drive motion of the movable component), the point at which the actuation element is coupled to a shaft (if the actuation element is to drive translation of the shaft), the point at which the cable is coupled to another actuation element (if the actuation element is to drive motion of the other actuation element), and so on. For example, in the embodiment of FIG. 5, the coupling points 565_2 to 565_5 are illustrated, which are fixed to the articulable structures 230. In other embodiments, the coupling points may be different. A coupling point can constitute an end point of a given segment of the actuation element, which may be, but does not necessarily have to be, an end point of the entire actuation element. For example, in the embodiment of FIG. 5, the coupling points 565_2 to 565_5 constitute end points of the entire actuation elements 500_2 to 500_5. In other embodiments, one or more coupling points are not the end point of their respective actuation element—for example, the coupling point may be a point where a portion of the actuation element meets a pulley attached to the component driven by the actuation element. Moreover, the operable coupling of the actuation element to the structure it drives at the coupling point does not necessarily entail the actuation element being fixedly attached to the structure. Instead, the actuation element may be movably coupled to the structure it drives, for example via a pulley attached to the structure as previously mentioned.

In some circumstances, multiple portions of an actuation element may be coupled to the same drive member or to separate drive members. For example, one end of the actuation element may be coupled to a drive member, extend from the drive member to the structure driven by the actuation element, and then loop back such that an opposite end of the actuation element is coupled to a drive member (the same or different drive member). In such cases, there are two segments of the actuation element that extend between the drive member(s) and the coupling point. In such circumstances, references to “paying out” or “drawing in” the actuation element should be understood as referring to paying in or drawing out one of these segments of the actuation element. The particular segment that is being referenced may be understood from the context if not explicitly identified.

Further, the terminology used herein to describe aspects of the invention, such as spatial and relational terms, is chosen to aid the reader in understanding example embodiments of the invention but is not intended to limit the invention. For example, spatially terms—such as “beneath”, “below”, “lower”, “above”, “upper”, “proximal”, “distal”, “up”, “down”, and the like—may be used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein may need to be interpreted differently in that different reference frame. For example, the direction referred to as “up” in relation to one of the figures may correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items may be posed differently.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

Unless otherwise noted herein or implied by the context, when terms of approximation such as “substantially,” “approximately,” “about,” “around,” “roughly,” and the like, are used in conjunction with a stated numerical value, property, or relationship, such as an end-point of a range or geometric properties/relationships (e.g., parallel, perpendicular, straight, etc.), this should be understood as meaning that mathematical exactitude is not required for the value, property, or relationship, and that instead a range of variation is being referred to that includes but is not strictly limited to the stated value, property, or relationship. In particular, the range of variation around the stated value, property, or relationship includes at least any inconsequential variations from the value, property, or relationship, such as variations that are equivalents to the stated value, property, or relationship. The range of variation around the stated value, property, or relationship also includes at least those variations that are typical in the relevant art for the type of item in question due to manufacturing or other tolerances.

As used herein, “transverse” refers to a positional relationship of two items in which one item is oriented crosswise at an angle relative to the other item, such as being substantially or generally perpendicular to the other item. As used herein, “transverse” includes, but does not require, an exactly perpendicular relationship. For example, unless otherwise noted herein or implied by the context, “transverse” may include at least positional relationships in which one item is oriented at nonparallel angle to the other item, such as for example, an angle ranging from 45° to 135° relative to the other item.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. A medical instrument comprising:
an instrument shaft comprising a proximal portion and a distal portion;
a first movable component coupled to the distal portion of the instrument shaft;
a first actuation element coupled to the instrument shaft;
a second actuation element;
a third actuation element; and
a transmission assembly movably coupled to the instrument shaft and comprising:
a first drive member, wherein at least one of the first, second, or third actuation elements is operably coupled to the first drive member and configured to drive translation of the instrument shaft relative to the transmission assembly in response to rotation of the first drive member; and
a second drive member comprising:
a rotatable drive shaft;
a first actuation transfer mechanism coupled to the rotatable drive shaft, the second actuation element being coupled to the first actuation transfer mechanism; and
a second actuation transfer mechanism coupled to the rotatable drive shaft, the third actuation element being coupled to the second actuation transfer mechanism;
wherein the first and second actuation transfer mechanisms are configured to actuate the second and third actuation elements in opposite directions as one another to drive a first degree of freedom of motion of the first movable component.

2. (canceled)

3. The medical instrument of claim 1, wherein the first and second actuation transfer mechanisms are configured such that:
in response to rotation of the rotatable drive shaft in a first direction, the first actuation transfer mechanism pays out the second actuation element while the second actuation transfer mechanism draws in the third actuation element; and
in response to rotation of the rotatable drive shaft in a second direction, the first actuation transfer mechanism draws in the second actuation element while the second actuation transfer mechanism pays out the third actuation element.

4. The medical instrument of claim 1, wherein the second and third actuation elements are operably coupled to the first drive member and the first and second actuation transfer mechanisms are configured to:
allow the first drive member to actuate the second and third actuation elements independently of rotation of the drive shaft of the second drive member; and
actuate the second and third actuation elements in response to rotation of the drive shaft of the second drive member independently of rotation of the drum of the first drive member.

5. (canceled)

6. The medical instrument of claim 1, further comprising:
a first pushable actuation element housed in the instrument shaft and operably coupled to the first movable component such that translation of the first pushable actuation element relative to the instrument shaft drives the first degree of freedom of motion of the first movable component,
wherein the second and third actuation elements are operably coupled to the first pushable actuation element such that
rotation of the drive shaft in the first direction causes translation of the first pushable actuation element relative to the instrument shaft in a proximal direction; and
rotation of the drive shaft in the second direction causes translation of the first pushable actuation element relative to the instrument shaft in a distal direction.

7. (canceled)

8. The medical instrument of claim 6, wherein:
the second actuation element extends proximally from the transmission assembly and is coupled to the first pushable actuation element at a first location that is proximal of the transmission assembly; and
the third actuation element extends distally from the transmission assembly and is coupled to the first pushable actuation element at a second location that is distal of the transmission assembly.

9. The medical instrument of claim 6, further comprising:
a second movable component;
a second pushable actuation element housed in the instrument shaft and operably coupled to the second movable component such that translation of the second pushable actuation element relative to the instrument shaft drives a second degree of freedom of motion of the second movable component;
a fourth actuation element operably coupled to the second pushable actuation element and to the first drive member;
a fifth actuation element operably coupled to the second pushable actuation element and to the first drive member; and
a third drive member configured to actuate the fourth and fifth actuation element to drive translation of the second pushable actuation element relative to the housing.

10. (canceled)

11. The medical instrument of claim 9, further comprising:
an end effector coupled to the instrument shaft;
wherein the first and second movable components are parts of the end effector.

12. The medical instrument of claim 11, wherein:
the first movable component comprises a jaw mechanism and the first degree of freedom of motion comprises at least one of opening or closing of the jaw mechanism; and
the second movable component comprises a translatable component and the second degree of freedom of motion comprises translation of the translatable component relative to the jaw mechanism.

13. The medical instrument of claim 12, wherein the jaw mechanism comprises electrosurgical electrodes.

14. The medical instrument of claim 9, wherein:
the fourth actuation element extends proximally from the transmission assembly and is coupled to the second pushable actuation element at a third location that is proximal of the transmission assembly; and
the fifth actuation element extends proximally from the transmission assembly to a pulley and extends distally from the pulley and is coupled to the second pushable actuation element at a fourth location that is proximal of the transmission assembly.

15. The medical instrument of claim 9, wherein the first pushable actuation element comprises an interior channel and the second pushable actuation element is coaxial with the first pushable actuation element and at least part of the second pushable actuation element extends through the interior channel of the first pushable actuation element.

16.-19. (canceled)

20. The medical instrument of claim 1, wherein:
each of the first, second, and third actuation elements is operably coupled to the first drive member and configured to drive translation of the instrument shaft relative to the transmission assembly in response to rotation of the first drive member.

21. The medical instrument of claim 1, wherein:
the first actuation element comprises a rotatable drum.

22. The medical instrument of claim 21, wherein:
a first subset of actuation elements, comprising at least the second and third actuation elements, are coupled to a distal portion of the instrument shaft;
a second subset of actuation elements, comprising at least the first actuation element, are coupled to a proximal portion of the transmission assembly; and
the first subset of actuation elements and the second subset of actuation elements are wound in opposite directions around the drum.

23. (canceled)

24. A medical instrument comprising:
an instrument shaft comprising a proximal portion and a distal portion;
a movable component supported by the distal portion of the instrument shaft;
a first pushable actuation element operably coupled to the movable component and translatable relative to the instrument shaft to drive a first degree of freedom of motion of the movable component;
a plurality of pullable actuation elements; and
a transmission assembly movably coupled to the instrument shaft and comprising:
a first drive member comprising a drum coupled to at least a subset of the plurality of pullable actuation elements and rotatable to actuate the subset of the plurality of pullable actuation elements to drive translation of the instrument shaft relative to the transmission assembly; and
a second drive member comprising a drive shaft rotatable to actuate a first pair of pullable actuation elements of the plurality of pullable actuation elements to drive translation of the pushable actuation element relative to the shaft.

25. The medical instrument of claim 24,
wherein the first pair of pullable actuation elements is operably coupled to the first drive member.

26. The medical instrument of claim 24, further comprising:
an end effector, wherein the movable component is part of the end effector.

27. The medical instrument of claim 26, further comprising:
a joint operably coupling the distal end portion of the instrument shaft to the end effector;
a third drive member comprising a second rotatable drive shaft, a second pair of pullable actuation elements of the plurality of pullable actuation elements being coupled to the second rotatable drive shaft to drive articulation of the joint.

28.-35. (canceled)

36. A method of operating a medical instrument comprising:
actuating a first drive member to simultaneously actuate a plurality of actuation elements coupled to the first drive member to drive relative translation between an instrument shaft and a transmission assembly of the medical instrument; and
actuating a second drive member to simultaneously actuate a first pair of actuation elements out of the plurality of actuation elements in opposite directions to drive motion of a movable component supported by the instrument shaft, wherein the first pair of actuation elements are coupled to both the first and second drive members.

37. The method of claim 36, wherein:
actuating the first drive member comprises actuating the first pair of actuation elements independently of actuation of the second drive member; and
actuating the second drive member comprises actuating the first pair of actuation elements independently of actuation of the first drive member.

38.-40. (canceled)
